(19) United States
(12) Patent Application Publication
(10) Pub. No.: US 2012/0156242 A1
(43) Pub. Date: Jun. 21, 2012

(54) An Antigenic Peptide Derived From Influenza Virus And A Method For Selecting Anti-Influenza Virus Antibody

(76) Inventors: Kazuyoshi Ikuta, Osaka (JP); Ritsuko Koketsu, Osaka (JP); Akifumi Yamashita, Osaka (JP); Norihito Kawashita, Osaka (JP); Mikihiro Yunoki, Osaka (JP); Yoshinobu Okuno, Kagawa (JP); Shoji Ideno, Osaka (JP); Motoki Kuhara, Nagano (JP)

(21) Appl. No.: 13/375,661

(22) PCT Filed: Jun. 16, 2010
(86) PCT No.: PCT/JP2010/003999

§ 371 Date: Mar. 6, 2012

Related U.S. Application Data
(60) Provisional application No. 61/187,702, filed on Jun. 17, 2009.

(51) Int. Cl. A61K 39/145; C07K 7/06; C07K 7/08; C07K 14/11; A61P 31/16; G01N 33/566; G01N 33/569; C07K 16/10; A61P 37/04; C07K 2/00; C07H 21/00

(52) U.S. Cl. 424/209.1; 530/300; 530/328; 530/327; 530/326; 530/324; 536/23.72; 435/7.92; 435/5; 436/501; 530/389.4

(57) ABSTRACT

Antigenic peptides are provided that can be used to induce global neutralizing antibodies, or antibodies reactive against a wide range of influenza A virus strains. The antigenic peptide can correspond to SEQ ID NO: 34 (EKEVLVLWG), SEQ ID NO: 2 (KFDKLYIWG), SEQ ID NO: 71(QEDLLVLWG), SEQ ID NO: 51 (EGRINYYWTLLEP), SEQ ID NO: 3 (PSRISIYWTIVKP), and/or SEQ ID NO: 82 (SGRMEFFWTILKP).

This application claims the benefit of prior U.S. Provisional Patent Application No. 61/187,702, filed Jun. 17, 2009, which is incorporated in its entirety by reference herein.

### TECHNICAL FIELD

This invention is concerning an antigenic peptide derived from influenza virus, a vaccine against influenza virus comprising the antigenic peptide and a method for selecting anti-influenza virus antibody.

### BACKGROUND ART

Influenza pandemics are rarely occurring but recurrent events. Such pandemics are associated with the emergence of new types of influenza virus for which the human population has no immunity. The highly pathogenic avian influenza A virus H5N1 was widely believed to be the most likely causative candidate for the next pandemic (NPL 1 (Carter and Plosker, 2008); NPL 2 (Pappaioanou, 2009)). However, novel swine-origin pandemic influenza A (H1N1) virus, named H1N1 pdm virus thereafter, has emerged in April, 2009 with a human pandemic potential (NPL 3 (Novel Swine-Origin Influenza A (H1N1) Virus Investigation Team, 2009); NPL 4 (Shinde et al., 2009)). Since it is not known beforehand which strain of influenza A virus could give rise to a next pandemic, prepandemic vaccines that can induce broadly cross-reactive immune responses are mandatory.

Influenza virus undergoes a high rate of antigenic changes giving rise to a new type of influenza strain(s). Influenza A viruses are classified into subtypes based on the antigenicity of their hemagglutinin (HA) and neuraminidase (NA) molecules; i.e., 16 HA (H1-H16) and 9 NA (N1-N9) subtypes (NPL 5; 6 (Fouchier et al., 2005; Webster et al., 1992)). Human influenza A viruses of at least three HA subtypes, H1, H2 and H3 have emerged as important pathogens. Currently, H1N1 and H3N2 influenza A viruses are seasonally circulating and causing human infections. In 2003, influenza virus subtype H5 of avian origin emerged as a human pathogen and it is much more lethal than earlier strains (NPL 7 (Webby and Webster, 2003)). Recently, the H1N1 virus emerged in April, 2009 as a novel type of influenza virus and is spreading rapidly in the human population.

Influenza infection can be more dangerous for certain groups of individuals, such as those having suffered from a heart attack patient or the elderly. A vaccine against influenza is therefore highly desirable. The influenza A virus contains in its membrane two highly immunogenic, but very variable proteins, HA and NA. The variable peptides in HA have disclosed in NPLs 9 and 10 (Webster and Laver, 1980 and Wiley et al., 1981).

Due to the variability of HA and NA, a vaccine with broad spectrum against influenza has so far not been developed. There are disclosed the membrane protein M2 and amino acid residues (aa) 16-23 of HA2 subunit as an immunoprotective antigen for use in vaccines with broad spectrum in PTL 1 and PTL 2. Novel epitope in influenza proteins which can be used for eliciting broad protection are much awaited for vaccine development.

The references listed below and all publications mentioned herein are incorporated in their entirety by reference herein.

### [Citation List]

### [Patent Literature]

PTL 1: Japanese Patent Application Laid-open No. JP 2009-22186

PTL 2: International Laid-open Patent Publication WO 99/07839

### [Non Patent Literature]

NPL 1: Carter, N. J., Plosker, G. L., 2008, “Prepandemic influenza vaccine H5N1 (split virion, inactivated, adjuvanted) [Prepandrix]: a review of its use as an active immunization against influenza A subtype H5N1 virus,” BioDrugs 22, 279-292.

NPL 2: Pappaioanou, M., 2009, “Highly pathogenic H5N1 avian influenza virus: cause of the next pandemic?” Comp. Immunol. Microbiol. Infect. Dis. 32, 287-300.

NPL 3: Novel Swine-Origin Influenza A (H1N1) Virus Investigation Team, 2009, “Emergence of a novel swine-origin influenza A (H1N1) virus in humans,” New Engl. J. Med. 361, June 18, 2009, pp.2605-2615

NPL 4: Shinde, V., Bridges, C. B., Uyeki, T. M., Shu, B., Balish, A., Xu, X., Lindstrom, S., Gubareva, L. V., Deyde, V., Garten, R. J., Harris, M., Gerber, S., Vagoski, S., Smith, F., Pascoe, N., Martin, K., Dufficy, D., Ritger, K., Conover, C., Quinlisk, P., Klimov, A., Bresee, J. S., Finelli, L., 2009, “Triple-reassortant swine influenza virus A (H1) in humans in the United States, 2005-2009,” New Engl. J. Med., Jun. 18, 2009 pp. 2616-2625

NPL 5: Fouchier, R. A., Munster, V., Wallenstein, A., Bestebroer, T. M., Herfst, S., Smith, D., Rimmelzwaan, G. F., Olsen, B., Osterhaus, A. D., 2005, “Characterization of a novel influenza A virus hemagglutinin subtype (H 16) obtained from black-headed gulls,” J. Virol. 79, 2814-2822.

NPL 6: Webster, R. G., Bean, W. J., Gorman, O. T., Chambers, T. M., Kawaoka, Y., 1992, “Evolution and ecology of influenza A viruses,” Microbiol. Rev. 56, 152-179.

NPL 7: Webby, R. J., Webster, R. G. 2003, “Are we ready for pandemic influenza?” Science 302, 1519-1522.

NPL8: Webster, R. G., Laver, W. G., 1980, “Determination of the number of nonoverlapping antigenic areas on Hong Kong (H3N2) influenza virus hemagglutinin with monoclonal antibodies and the selection of variants with potential epidemiological significance,” Virology 104, 139-148.

NPL9: Wiley, D. C., Wilson, I. A., Skehel, J. J., 1981, “Structural identification of the antibody-binding sites of Hong Kong influenza haemagglutinin and their involvement in antigenic variation,” Nature 289, 373-378.

### SUMMARY OF INVENTION

### [Technical Problem]

The problem to be solved is to provide a novel immunoprotective antigenic peptide and vaccines comprising the peptide.

### [Solution to Problem]

The present invention relates to antigenic peptides that can be used to induce global neutralizing antibodies or antibodies reactive against a wide range of influenza A virus strains. The antigenic peptides can comprise an upper and/or a lower region, for example, SEQ ID NO: 2 and/or SEQ ID NO: 3. SEQ ID NO: 2 and SEQ ID NO: 3 are present in HA of subtype H3N2 and can be highly conserved sequences in a wide range of H3N2 virus strains. Further, sequences corresponding to these two epitope regions can be highly conserved in all influenza A viruses belonging to the same subtype. For example, sequences in other influenza A subtypes such as H1N1 including H1N1 pdm, and H5N1 derived from human, swine and/or avian that correspond to the above two epitope regions of human H3N2, can be conserved in the same subtypes. These sequences can be conserved between strains derived from different hosts, for example, human, swine, or avian. Especially, an antibody reactive to the two epitope regions of a certain strain within a certain subtype is expected to neutralize other strains within the same subtype. The two epitope regions of not only H3N2 but also other subtypes such as H1N1 and H5N 1 can function as a significant human epitope to induce global neutralizing antibodies.

It is therefore a feature of the present invention to provide an antigenic peptide to induce global neutralizing antibodies.

Another feature of the present invention is to provide a vaccine against influenza A virus comprising at least one kind of antigenic peptide.

A further feature of the present invention is to select an antibody or antibodies that can recognize influenza A subtypes.

Additional features and advantages of the present invention will be set forth in part in the description that follows, and in part will be apparent from the description, or can be learned by practice of the present invention. The objectives and other advantages of the present invention will be realized and attained by means of the elements and combinations particularly pointed out in the description and appended claims.

To achieve these and other advantages, and in accordance with the purposes of the present invention, as embodied and broadly described herein, the present invention relates to an antigenic peptide comprising at least an amino acid sequence of an upper region and/or a lower region of beta sheet structure within hemagglutinin HA1 region of influenza A virus.

The present invention further relates to an isolated nucleic acid molecule comprising a nucleotide sequence of a gene encoding the antigenic peptide of the present invention.

The present invention further relates to a vaccine against influenza A virus comprising at least one antigenic peptide, a reagent for influenza test comprising at least one antigenic peptide, a reagent kit for influenza test comprising at least one antigenic peptide, and a method for selecting an antibody that recognizes an upper region and/or a lower region of beta sheet structure within hemagglutinin HA1 region of an influenza A virus subtype.

It is to be understood that both the foregoing general description and the following detailed description are exemplary and explanatory only and are intended to provide a further explanation of the present invention, as claimed.

The accompanying drawings, which are incorporated in and constitute a part of this application, illustrate aspects of the present invention and together with the description, serve to explain the principles of the present invention.

### [Advantageous Effect of Invention]

The peptide of the present invention has strong immunogenicity. Specifically, the peptide can elicit an antibody in vivo, which have reactivity to the peptide, and stimulate human PBMCs to neutralize the influenza virus. Especially, an antibody, which is elicited by the peptide derived from one strain within one subtype, is expected to neutralize other strains belonging to the same subtype. It is expected that the vaccine comprising the peptide of the present invention can effectively stimulate the immune system in living organisms. An antibody obtained by the selecting method of the present invention can effectively protect living organism from the influenza virus.

### BRIEF DESCRIPTION OF DRAWINGS

FIG. 1 is a sequence logo of the known neutralizing epitopes A-E of human influenza A virus H3N2.

FIG. 2A is a sequence variation and accumulation rates at the upper and the lower region in H1N1 derived from different hosts.

FIG. 2B is a sequence variation and accumulation rates at the upper and the lower region in H3N2 and H5N1 derived from different hosts.

FIG. 3 is a sequence logo of the upper and the lower regions of various subtypes of influenza A virus.

FIGS. 4A, 4B, and 4C show the evolutionary relationships of HA1 region and the upper and the lower regions of various subtypes of influenza A virus by ME method.

FIG. 5A shows the upper regions and the upper parts comprising the upper regions from various strains of H3N2 subtype, amino acid sequences from aa 167-187. FIG. 5B shows the lower regions and the lower parts comprising the lower regions from various strains of H3N2 subtype, amino acid sequences from aa 225-241.

FIG. 6A shows the reaction of antiserum to the upper peptide or the lower peptide of H1N1.

FIG. 6B shows the reaction of antiserum to the upper peptide or the lower peptide of H3N2.

FIG. 6C shows the reaction of antiserum to the upper peptide or the lower peptide of H5N1.

FIG. 7A shows the antibody titer of rabbit immune serum against the peptide of the upper part of H3N2.

FIG. 7B shows the antibody titer of rabbit immune serum against the peptide of the lower part of H3N2.

FIG. 7C shows the antibody titer of rabbit immune serum against HA vaccine.

FIGS. 8A-8B shows neutralization inhibition by the synthetic peptides.

FIG. 9 shows neutralization test with culture supernatant of human PBMCs stimulated with the synthetic peptides.

FIG. 10 shows neutralization test with culture supernatant of human PBMCs stimulated with the synthetic peptides.

### DESCRIPTION OF EMBODIMENT

The present invention relates to an antigenic peptide, or a polypeptide that can be used to induce neutralizing antibodies reactive against a wide-range of influenza virus strains. The antigenic peptide in the present invention comprises an amino acid sequence corresponding to an upper region and/or a lower region of beta sheet structure within HA1 region of influenza A virus subtype. The antigenic peptide of the present invention may comprise either the upper region or the lower region, or both regions.

“An upper region” of beta sheet structure within HA1 region of influenza A virus means region of the amino acid sequence corresponding to aa 173-181 (SEQ ID NO: 2) in amino acid sequence of SEQ ID NO: 1, HA of A/Hiroshima/52/2005(H3N2) strain. “A lower region” of beta sheet structure within HA1 region of influenza A virus means region of the amino acid sequence corresponding to aa 227-239 (SEQ ID NO: 3) in amino acid sequence of SEQ ID NO: 1, HA of A/Hiroshima/52/2005(H3N2) strain. The upper region is selected from the group consisting of aa 173-181 in HA of A/Hiroshima/52/2005(H3N2) strain (SEQ ID NO:1) , the corresponding regions in other strains, the corresponding regions in other subtypes, and their variants. The lower region is selected from the group consisting of aa 227-239 in HA of A/Hiroshima/52/2005(H3N2) strain (SEQ ID NO:1), the corresponding regions in other strains, the corresponding region in other subtypes, and their variants. For example, the upper region corresponds to aa 179-187 in amino acid sequence of GenBank Accession No. BAA08716 (A/Guizhou/54/1989 strain), aa 189-197 in amino acid sequence of GenBank Accession No. AAT08000 (A/Wyoming/3/2003 strain), or aa 173-181 in amino acid sequence of GenBank Accession No. ACC66685 (A/New York/55/2004 strain), and the lower region corresponds to aa 233-245 in amino acid sequence of GenBank Accession No. BAA08716 (A/Guizhou/54/1989 strain), aa 243-255 in amino acid sequence of GenBank Accession No. AAT08000 (A/Wyoming/3/2003 strain), or aa 227-239 in amino acid sequence of GenBank Accession No. ACC66685 (A/New York/55/2004 strain).

The upper region and/or the lower region of H3N2 can be highly conserved sequences in a wide range of H3N2 virus strains. Further, the sequences corresponding to these two regions in other subtype can be highly conserved in all influenza A viruses belonging to the same subtype. For example, sequences that correspond to the above two regions of human H3N2, can be identified in other influenza A subtypes such as H1N1 including H1N1 pdm, and H5N1 derived from human, swine and/or avian. The upper and the lower regions within HA of influenza A virus H3N2 form the anti-parallel beta-sheet structure and other subtypes, such as H1N1 and H5N1, can have corresponding regions with similar structure. Therefore, an antibody reactive to these epitope regions of human H3N2 is expected to neutralize other influenza A viruses such as H1N1, and H5N1. Especially, an antibody reactive to the two epitope regions of one strain belonging to one subtype is expected to neutralize other strains belonging to the same subtype. The two epitope regions of not only H3N2 but also other subtypes such as HIN1 and H5N1 can function as a significant human epitope to induce neutralizing antibodies in individual subtypes.

The antigenic peptide can comprise an amino acid sequence that corresponds to aa 173-181 (SEQ ID NO: 2) that forms the anti-parallel beta-sheet upper region within HA of influenza A virus H3N2 and/or aa 227-239 (SEQ ID NO: 3) that forms the anti-parallel beta-sheet lower region within HA of influenza A virus H3N2. The amino acid sequence of the upper region can comprise SEQ ID NO: 34 (EKEVLVLWG) (H1N1), SEQ ID NO: 2 (KFDKLYIWG) (H3N2), SEQ ID NO: 71(QEDLLVLWG) (H5N1) or their variants; and the amino acid sequence of the lower region can comprise SEQ ID NO: 51 (EGRINYYWTLLEP) (H1N1), SEQ ID NO: 3 (PSRISIYWTIVKP) (H3N2), SEQ ID NO: 82 (SGRMEFFWTILKP) (H5N1) or their variants. The variants of the region comprising SEQ ID NO: 2 include, for example, amino acid sequence selected from SEQ ID NOs: 34-50 (H1N1), 8-20 (H3N2), 71-81 (H5N1), 177 (H1N1), 179 (H3N2), and 181 (H5N1). The variants of the region comprising SEQ ID NO: 3 include, for example, amino acid sequence selected from SEQ ID NOs: 51-70 (H1N1), 21-33 (H3N2), 82-96 (H5N1), 178 (H1N1), 180 (H3N2), and 182 (H5N1).

The antibody against the amino acid sequence of the upper region and/or the lower region is expected to neutralize all influenza A virus subtypes. The influenza A virus can comprise an H1, H2, H3, H4, H5, H6, H7, H8, H9, H10, H11, H12, H13, H14, H15, or H16 subtype. The influenza A virus can comprise H1N1, H1N1 pdm, H3N2, or H5N1 subtypes. Each subtype includes many different strains due to the variability of the HA region. For example, H3N2 subtype includes A/Hiroshima/52/2005 strain (GenBank Accession No. EU501660), A/Aichi/2/1968 strain, A/Guizhou/54/1989 strain (GenBank Accession No. D49963), A/Wyoming/3/2003 strain (GenBank Accession No. AY531033), A/New York/55/2004 strain (GenBank Accession No. EU501486). The antibody against the amino acid sequence of the upper region and/or the lower region is expected to neutralize all influenza A virus strains derived from human, swine, avian, or other hosts belonging to the same subtype.

An amino acid sequence of an upper part of an influenza A virus subtype comprises the upper region. An amino acid sequence of a lower part of an influenza A virus subtype comprises the lower region. The antigenic peptide of the present invention may comprise either the upper part or the lower part, or both parts.

The upper part comprising the upper region means the part of the amino acid sequence corresponding to aa 167-187 (SEQ ID NO: 4) in amino acids sequence of SEQ ID NO: 1, HA of A/Hiroshima/52/2005(H3N2) strain. The lower part comprising the lower region means the part of the amino acid sequence corresponding to aa 225-241 (SEQ ID NO: 5) in the amino acids sequence of SEQ ID NO: 1, HA of A/Hiroshima/52/2005(H3N2) strain. The upper part is selected from the group consisting of aa 167-187 in HA of A/Hiroshima/52/2005(H3N2) strain (SEQ ID NO:1) , the corresponding parts in other strains, the corresponding parts in other subtypes, and their variant. The lower part is selected from the group consisting of aa 225-241 in HA of A/Hiroshima/52/2005(H3N2) strain (SEQ ID NO:1), the corresponding parts in other strains, the corresponding parts in other subtypes, and their variant. The variants of SEQ ID NO: 4 or 5 include the amino acid sequence of the upper or the lower part in other subtype such as H1N1 or H5N1. The variants of SEQ ID NO: 4 or 5 can include, for example, the amino acid sequences of SEQ ID NO: 6 or 7.

The upper part can be an amino acid sequence corresponding to aa 167-187 (SEQ ID NO: 4) present at a position from N-terminus in HA1 region of human H3N2 of the influenza A virus and the lower part of influenza A human H3N2 virus can be aa 225-241 (SEQ ID NO: 5) present at a position from N-terminus in HA1 region of human H3N2 of the influenza A virus.

The present invention also relates to a vaccine against influenza A virus comprising at least one antigenic peptide identified herein. The antigenic peptide can be obtained by the known method, for example, the genetic recombination technique utilizing well-known hosts such as Escherichia coli and retrovirus, or the method known in the general peptide chemistry. “Peptide Synthesis, Maruzen, 1975” and “Peptide Synthesis, Interscience, New York 1996” can be exemplified, but known methods are widely available. Peptides according to the present invention can be purified/collected by the combination of the gel filtration chromatography, the ion column chromatography, the affinity chromatography, and the like using the recognition property. A method to specifically adsorb/collect a peptide by a polyclonal antibody or monoclonal antibody, that is prepared against the peptide, is more preferably used.

The antigenic peptides can induce neutralizing antibodies reactive against a wide-range of influenza virus strains. The vaccine of the present invention can be prepared by synthesizing or isolating a polypeptide according to the invention, and solubilizing or dispersing the polypeptide in a medium, and optionally adding other influenza virus antigens and/or a carrier, vehicle and/or adjuvant substance. The vaccine according to the present invention can comprise at least two different antigenic peptides or polypeptide fragments. The vaccine comprise preferably the antigenic peptide comprising the upper region and the antigenic peptide comprising the lower region, and more preferably the antigenic peptide comprising the upper part and the antigenic peptide comprising the lower part.

Preparation of vaccines which contain the peptide as active ingredients is generally well understood in the art, as exemplified by U.S. Pat. No. 4,608,251; 4,601,903; 4,599,231; 4,599,230; 4,596,792; and 4,578,770, all incorporated herein by reference. Typically, such vaccines are prepared as injectables either as liquid solutions or suspensions; solid forms suitable for solution in, or suspension in, liquid prior to injection can also be prepared. The preparation can also be emulsified. The active immunogenic ingredient is often mixed with excipients which are pharmaceutically acceptable and compatible with the active ingredient. Suitable excipients are, for example, water, saline, dextrose, glycerol, ethanol, or the like, and combinations thereof. In addition, if desired, the vaccine can contain minor amounts of auxiliary substances such as wetting or emulsifying agents, pH buffering agents, or adjuvants which enhance the effectiveness of the vaccines.

Seed viruses for the production of inactivated influenza virus, such as influenza A H1N1 and H3N2 viruses as well as influenza B virus, are naturally occurring virus strains that replicate well in the allantoic cavity of embryonated chicken eggs. Further, a similar vaccine against the highly pathogenic H5N1 virus could be prepared by reverse genetics of H5N 1-derived HA and NA genes, with the internal genes of a high-yield strain in order to allow well growth of the H5 virus in chicken embryos (Carter and Plosker, 2008; Takahashi, Y., Hasegawa, H., Hara, Y., Ato, M., Nonomiya, A., Takagi, H., Odagiri, T., Sata, T., Tashiro, M., Kobayashi, K., 2009, “Protective immunity afforded by inactivated H5N1 (NIBRG-14) vaccine requires antibodies against both hemagglutinin and neuraminidase in mice,” J. Infect. Dis. 199, 1629-1637). The peptide of the present invention may be isolated from inactivated virus which is obtained by the above method.

The vaccines can be conventionally administered parenterally, by injection, for example, either subcutaneously or intramuscularly. Additional formulations which are suitable for other modes of administration include suppositories and, in some cases, oral formulations.

For suppositories, traditional binders and carriers can include, for example, polyalkylene glycols or triglycerides; such suppositories can be formed from mixtures containing the active ingredient in the range of 0.56 to 10%, preferably 1-2% by weight of total weight of formulation. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10-95% by weight of active ingredient, preferably 25-70% by weight of total weight of formulation.

The polypeptides or proteins can be formulated into the vaccine as neutral or salt forms. Pharmaceutically acceptable salts include acid addition salts (formed with the free amino groups of the peptide) and which are formed with inorganic acids such as, for example, hydrochloric or phosphoric acids, or such organic acids as acetic oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 microgram to 1000 microgram, such as in the range from about 1 microgram to 300 microgram, and especially in the range from about 10 microgram to 50 microgram. Suitable regimens for initial administration and booster shots are also variable but are typified by an initial administration followed by subsequent inoculations or other administrations.

The manner of application can be varied widely. Any of the conventional methods for administration of a vaccine are applicable. These are believed to include oral application on a solid physiologically acceptable base or in a physiologically acceptable dispersion, parenterally, by injection or the like. The dosage of the vaccine will depend on the route of administration and will vary according to the age of the person to be vaccinated and, to a lesser degree, the size of the person to be vaccinated.

Some of the polypeptides of the vaccine are sufficiently immunogenic in a vaccine, but for some of the others the immune response will be enhanced if the vaccine further comprises an adjuvant substance.

Various methods of achieving adjuvant effect for the vaccine include use of agents such as aluminum hydroxide or phosphate (alum), commonly used as 0.05 to 0.1 percent solution in phosphate buffered saline, admixture with synthetic polymers of sugars (Carbopol) used as 0.25 percent solution, aggregation of the protein in the vaccine by heat treatment with temperatures ranging between 70 to 101 degrees C. for 30 seconds to 2 minutes periods respectively. Aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide monooleate (Aracel A) or emulsion with 20 percent solution of a perfluorocarbon (Fluosol-DA) used as a block substitute can also be employed. According to the invention, DDA (dimethyldioctadecylammonium bromide) is an example of an adjuvant. Freund's complete and incomplete adjuvants, as well as QuilA and RIBI can be used as adjuvants. Further examples are monophosphoryl lipid A (MPL), and muramyl dipeptide (MDP).

Another way to achieve adjuvant effect is to employ the technique described in Gosselin et al., 1992 (which is hereby incorporated by reference herein). In brief, the presentation of a relevant antigen such as an antigen of the present invention can be enhanced by conjugating the antigen to antibodies (or antigen binding antibody fragments) against the Fcz receptors on monocytes/macrophages. Especially conjugates between antigen and anti-FceRI have been demonstrated to enhance immunogenicity for the purposes of vaccination.

Other ways involve the use of immune modulating substances such as lymphokines (e.g. IFN-gamma, IL-2 and IL-12) or synthetic IFN-gamma inducers such as poly I:C in combination with the above-mentioned adjuvants.

Multiple administrations of the vaccine can be used, usually not exceeding six vaccinations, more usually not exceeding four vaccinations and preferably one or more, usually at least about three vaccinations. The vaccinations will normally be at from two to twelve week intervals, more usually from three to five week intervals, though other intervals can be used. Periodic boosters at intervals of 1-5 years, such as three years, can be desirable to maintain the desired levels of protective immunity. The course of the immunization can be followed by in vitro proliferation assays of PBL (peripheral blood lymphocytes) or PBMC co-cultured with the polypeptide of the present invention, and especially by measuring the levels of IFN released from the primed lymphocytes. The assays can be performed using conventional labels, such as radionuclides, enzymes, fluorescers, and the like. These techniques are well known and can be found in a wide variety of patents, such as U.S. Pat. Nos. 3,791,932; 4,174,384 and 3,949,064, as illustrative of these types of assays.

Due to genetic variation, different individuals can react with immune responses of varying strength to the same polypeptide. Therefore, the vaccine according to the invention can comprise several different polypeptides (one or more different polypeptides) in order to increase the immune response. The polypeptides can either act due to their own immunogenicity or merely act as adjuvants.

The present invention also relates to a method for immunizing an animal, such as a human being, comprising administering to the animal the polypeptide of the invention, or a vaccine composition of the invention. Preferred routes of administration are the parenteral (such as intravenous and intraarterially), intraperitoneal, intramuscular, subcutaneous, intradermal, oral, buccal, sublingual, nasal, rectal or transdermal route.

The present invention also relates to an isolated nucleic acid molecule comprising a nucleotide sequence of a gene encoding the antigenic peptide. For example, the nucleotide sequence is selected from GenBank Accession No. EU501660 (A/Hiroshima/52/2005 strain), GenBank Accession No. D49963 (A/Guizhou/54/1989 strain), GenBank Accession No. AY531033 (A/Wyoming/3/2003 strain), GenBank Accession No. EU501486 (A/New York/55/2004 strain). The nucleotide sequence may comprise the nucleotide sequence selected from nucleotide sequences coding for the upper and/or the lower region as described below.

(1) Nucleotide sequences coding for the upper region
(i) AAATTTGACAAATTGTACATTTGGGGG (SEQ ID NO: 173) (517-543 in a nucleotide sequence of GenBank Accession No. EU501486)
(ii) AAATTTGACAAATTGTACATTTGGGGG (SEQ ID NO: 173) (565-591 in a nucleotide sequence of GenBank Accession No. AY531033)
(iii) AAATTTGACAAATTGTACATTTGGGGG (SEQ ID NO: 173) (537-563 in a nucleotide sequence of GenBank Accession No. D49963)
(iv) AAATTTGACAAATTGTACATTTGGGGG (SEQ ID NO: 173) (517-543 in a nucleotide sequence of GenBank Accession No. EU501660)
(2) Nucleotide sequences coding for the lower region
(i) TCCAGCAGAATAAGCATCTATTGGACAATAGTAAAACCG (SEQ ID NO: 174) (727-765 in a nucleotide sequence of GenBank Accession No. AY531033)
(ii) TCTAGTAGAATAAGCATCTATTGGACAATAGTAAAACCG (SEQ ID NO: 175) (699-737 in a nucleotide sequence of GenBank Accession No. D49963)
(iii) CCCAGCAGAATAAGCATCTATTGGACAATAGTAAAACCG (SEQ ID NO: 176) (679-717 in a nucleotide sequence of GenBank Accession No. EU501660)
(iv) CCCAGCAGAATAAGCATCTATTGGACAATAGTAAAACCG (SEQ ID NO: 176) (679-717 in a nucleotide sequence of GenBank Accession No. EU501486)

The present invention also relates to a reagent for an influenza test comprising at least one kind of antigenic peptide. The reagent may comprise any component in addition to the antigenic peptide as long as the reagent is used for an influenza test.

The present invention also relates to a reagent kit for an influenza test comprising at least one kind of antigenic peptide. The reagent kit may comprise any component, reagent, and/or instruments in addition to the antigenic peptide as long as the reagent kit is used for an influenza test.

The present invention also relates to a method for selecting an antibody or antibodies which recognizes the upper region and/or the lower region of beta sheet structure within HA1 region of influenza A virus. The upper region can correspond to aa 173-181 (SEQ ID NO: 2) present at a position from N-terminus in HA1 region of human H3N2 of the influenza A virus and the lower region can correspond to aa 227-239 (SEQ ID NO: 3) present at a position from N-terminus in HA1 region of human H3N2 of the influenza A virus. The amino acid sequence of the upper region can comprise SEQ ID NO: 34 (EKEVLVLWG), SEQ ID NO: 2 (KFDKLYIWG), and/or SEQ ID NO: 71 (QEDLLVLWG), and the amino acid sequence of the lower region can comprise SEQ ID NO: 51 (EGRINYYWTLLEP), SEQ ID NO: 3 (PSRISIYWTIVKP), and/or SEQ ID NO: 82 (SGRMEFFWTILKP). The amino acid sequence of the upper region and/or the lower region can be highly conserved in all influenza A virus subtypes derived from human, swine, or avian hosts. The antibody can recognize the upper region of human H3N2 virus from an upper part of human H3N2 virus and/or the lower region of human H3N2 virus from a lower part human H3N2 virus. The upper part can correspond to aa 167-187 present at a position from N-terminus in HA1 region of human H3N2 of the influenza A virus and the lower part can correspond to aa 225-241 present at a position from N-terminus in HA1 region of human H3N2 of the influenza A virus. The influenza A virus can comprise an H1, H2, H3, H4, H5, H6, H7, H8, H9, H10, H11, H12, H13, H14, H15, or H16 subtype. The influenza A virus can comprise H1N1, H1N1 pdm, H3N2, or H5N1 subtypes. The method for selecting an antibody or antibodies can comprise incubating an antibody as a candidate with a peptide to detect the binding between the antibody and the peptide, wherein the peptide can comprise an amino acid sequence of the upper region and/or the lower region of beta sheet structure within HA1 region of influenza A virus. The method for selecting an antibody or antibodies can comprise incubating an antibody as a candidate with a peptide to detect the binding between the antibody and the peptide, wherein the peptide can comprise an amino acid sequence of the upper part and/or the lower part of beta sheet structure within HA 1 region of influenza A virus. The selected antibody can show neutralization of the influenza virus. The selected antibody can show inhibition of hemagglutination (HI).

Epitope sequences within HA of influenza A virus H3N2 at aa173-181 (SEQ ID NO: 2) and aa 227-239 (SEQ ID NO: 3) that forms anti-parallel beta-sheet structure can be recognized by HuMAbs, B-1 and D-1 (Kubota-Koketsu, R., Mizuta, H., Ohshita, M., Ideno, S., Yunoki, M., Kuhara, M., Yamamoto, N., Okuno, Y., Ikuta, K., “Broad neutralizing human monoclonal antibodies against influenza virus from vaccinated healthy donors,” Submitted on Jun. 19, 2009 and published on July 4). In particular, HuMAbs from the two hybridoma cell clones producing HuMAbs can have a high neutralizing activity against a wide range of H3N2 virus strains. There can be significant conservation of the beta-sheet region having or including the two epitope regions in H3N2 at aa 173-181 (SEQ ID NO: 2) and aa 227-239 (SEQ ID NO: 3). Other subtypes, such as H1N1 and H5N1, can have corresponding regions with similar structure. The two regions can be located underneath the receptor binding sites of these subtypes. Analysis of those regions using sequences available from the Influenza Virus Resource at the National Center for Biotechnology Information revealed that compared with known neutralizing epitopes A-E, these sequences are fairly conserved in human H3N2 (n=2594) and swine H1N1 (n=188) and H3N2 (n=95), and highly conserved in human H1N1 (n=1171) and recent swine-origin H1N1 pdm (n=312), as well as human H5N1 (n=224) and avian H5N1 (n=1534). Phylogenetic tree for these regions formed clearly separable clusters for H1N1, H3N2 and H5N1, irrespective of the host of origin. These regions can be used to develop vaccines that can induce neutralizing antibodies reactive against a wide-range of influenza virus strains.

Computer-based characterization of the H3N2 viruses also revealed relatively high conservation of two epitope regions. Further, the present inventors identified corresponding sequences in influenza viruses, such as H1N1 and H5N1 of different hosts that showed much higher conservation in their sequences than those in H3N2. These data allow for alternative vaccines against influenza A virus that could neutralize a wide range of virus strains.

The present inventors have focused on the upper and the lower regions that form anti-parallel beta-sheet structure within HA of human-derived H3N2 that are recognized by B-1 and D-1 HuMAbs. Interestingly, although the overall amino acid sequence of HA is poorly conserved among subtypes, the structure and functions of such HAs are highly conserved in individual subtypes. This indicates that although a case of evolution and sequence variation proceed to an extreme level, structure and functions have remained conserved (Palese, P., Shaw, M. L., 2007, Chapter 47, “Orthomyxoviridae: The viruses and their replication,” In: Knipe, D. M., Howley, P. M., Griffin, D. E A., Lamb, R. A., Martin, M. A., Roizman, B., Straus, S. E., (Ed.), Fields Virology; Wolters Kluwer: Lippincott Williams & Wilkins, 5^{th} edition, pp. 1647-1689). Most of the major neutralization epitopes were shown to be related to the former. Although the anti-parallel beta-sheet structure underneath the receptor-binding region is one of the human neutralizing epitope, the regions can also fulfill the latter category. As it was found that the regions are relatively highly conserved, this structure can play a role for viral infection.

It should be understood that any of the amino acid sequences described herein can further comprise any sequence having at least about 70%, preferably at least about 80%, more preferably at least about 90%, most preferably at least about 95% homology thereto (such as 95% to 98%, 98% to 99%, or about 99% homology). Further, the peptides of the present invention can comprise fragments of the peptides described herein and/or mutations in the peptides, such as deletions, insertions or substitutions of one or more amino acids (such as 1 to 25, 2 to 20, 3 to 15, 5 to 10 amino acids), either naturally occurring or artificially induced, either randomly or in a targeted fashion. For example, the peptide can comprise fragments of the peptides in which cysteine is added at its N-terminus or C-terminus. In the present invention, “variant” means the peptide as described above.

Human epitope regions to form anti-parallel beta-sheet structure within HA of human-derived H3N2 (the upper and the lower regions) that are recognized by B-1 and D-1 HuMAbs can show neutralizing activities to wide-range of strains in this subtype, but are not cross-reactive to H1N1 and B subtypes (Kubota-Koketsu et al.). To examine the sequence conservation rate of the two regions, 2594 complete HA sequences derived from human H3N2 were obtained that were available at May 15, 2009 from the Influenza Virus Resource at the National Center for Biotechnology Information (Bao, Y., Bolotov, P., Dernovoy, D., Kiryutin, B., Zaslaysky, L., Tatusova, T., Ostell, J., Lipman, D., 2008, “The Influenza Virus Resource at the National Center for Biotechnology Information,” J. Virol. 82, 596-601). Among these, 16 and 26 variants for the upper and the lower regions, respectively, were detected. This indicates that they produced one variant per 162.1 and 99.8 strains in these regions, respectively, as shown in Table 1 below.

TABLE 1
Ratio of appearance for the sequence variants in individual epitopes.

| | Host | | Sequence | Variant | Sequences |
|---|---|---|---|---|---|
| Subtype | origin | Epitope | number | number | per variant |
| H3N2 | Human | Epitope A | 2594 | 173 | 15.0 |
| | | Epitope B1 | 2594 | 49 | 52.9 |
| | | Epitope B2 | 2594 | 76 | 34.1 |
| | | Epitope C1 | 2594 | 33 | 78.6 |
| | | Epitope C2 | 2594 | 26 | 99.8 |
| | | Epitope D | 2594 | 41 | 63.3 |
| | | Epitope E | 2594 | 49 | 52.9 |
| | | Upper | 2594 | 16 | 162.1 |
| | | Lower | 2594 | 26 | 99.8 |
| | Swine | Upper | 95 | 11 | 8.6 |
| | | Lower | 95 | 8 | 11.9 |
| H1N1 | Human | Upper | 1171 | 11 | 106.5 |
| | | Lower | 1171 | 18 | 65.1 |
| | Swine | Upper | 188 | 14 | 13.4 |
| | | Lower | 188 | 19 | 9.9 |
| | Swine (pdm) | Upper | 312 | 1 | 312.0 |
| | | Lower | 312 | 2 | 156.0 |
| H5N1 | Human | Upper | 224 | 6 | 37.3 |
| | | Lower | 224 | 11 | 20.4 |
| | Avian | Upper | 1534 | 26 | 59.0 |
| | | Lower | 1534 | 32 | 47.9 |

In contrast, sequences of the known neutralizing epitopes A to E (Webster and Laver, 1980; Wiley et al., 1981) that were extracted from the same 2594 sequences were much more variable, especially in epitopes A and B2, i.e., one variant production per 15.0 and 34.1 strains, respectively (Table 1). Epitope C2 and the lower region showed the same frequency for variant production. However, this epitope C2 consisted of only 5 amino acids, while the lower region consisted of 13 amino acids, indicating much higher conservation in the sequences of this lower region.

In Table 2, the sequences of the top 10 variants and the percentages of individual variants in the known neutralizing epitopes A to E are comparatively shown.

TABLE 2
Amino acid variation of the known neutralizing epitopes.

| | | Sequence | Appearance | Cumulative |
|---|---|---|---|---|
| Epitope | Sequence | number | ratio (%) | ratio (%) |
| Epitope | NNESFNWTGVTQNGTSSACKRRSNNS (SEQ ID No. 97) | 421 | 16.2 | 16.2 |
| A | ...................I...... (SEQ ID No. 98) | 329 | 12.7 | 28.9 |
| (n = 2594) | ..........A............DK. (SEQ ID No. 99) | 243 | 9.4 | 38.3 |
| | ..........A.............K. (SEQ ID No. 100) | 201 | 7.7 | 46.0 |
| | .....D..................K. (SEQ ID No. 101) | 199 | 7.7 | 53.7 |
| | ..........A............IK. (SEQ ID No. 102) | 180 | 6.9 | 60.6 |
| | ........................K. (SEQ ID No. 103) | 89 | 3.4 | 64.1 |
| | I..D......A.D.K.Y....G.V.. (SEQ ID No. 104) | 84 | 3.2 | 67.3 |
| | ........................S. (SEQ ID No. 105) | 82 | 3.2 | 70.5 |
| | ..........A.....Y......IK. (SEQ ID No. 106) | 61 | 2.4 | 72.8 |
| Epitope | THLKFKYPA (SEQ ID No. 107) | 905 | 34.9 | 34.9 |
| B1 | HQ..Y.... (SEQ ID No. 108) | 728 | 28.1 | 63.0 |
| (n = 2594) | ....Y.... (SEQ ID No. 109) | 360 | 13.9 | 76.8 |
| | HK.EY.... (SEQ ID No. 110) | 261 | 10.1 | 86.9 |
| | ..S...... (SEQ ID No. 111) | 74 | 2.9 | 89.7 |
| | .KSGST..V (SEQ ID No. 112) | 55 | 2.1 | 91.9 |
| | HQ..YR... (SEQ ID No. 113) | 41 | 1.6 | 93.4 |
| | YKSGST..V (SEQ ID No. 114) | 38 | 1.5 | 94.9 |
| | HESEY.... (SEQ ID No. 115) | 19 | 0.7 | 95.6 |
| | YESES...V (SEQ ID No. 116) | 15 | 0.6 | 96.2 |
| Epitope | GTDSDQISLYAQ (SEQ ID No. 117) | 615 | 23.7 | 23.7 |
| B2 | ...N...F.... (SEQ ID No. 118) | 517 | 19.9 | 43.6 |
| (n = 2594) | S........... (SEQ ID No. 119) | 341 | 13.1 | 56.8 |
| | ...N........ (SEQ ID No. 120) | 303 | 11.7 | 68.5 |
| | S.....T..... (SEQ ID No. 121) | 146 | 5.6 | 74.1 |
| | S.....T...VR (SEQ ID No. 122) | 137 | 5.3 | 79.4 |
| | S.....T...V. (SEQ ID No. 123) | 102 | 3.9 | 83.3 |
| | ..YN........ (SEQ ID No. 124) | 68 | 2.6 | 85.9 |
| | S.NQE.T...V. (SEQ ID No. 125) | 59 | 2.3 | 88.2 |
| | ..NN........ (SEQ ID No. 126) | 47 | 1.8 | 90.0 |
| Epitope | GICDSPHQ (SEQ ID No. 127) | 1005 | 38.7 | 38.7 |
| C1 | R....... (SEQ ID No. 128) | 528 | 20.4 | 59.1 |
| (n = 2594) | E....... (SEQ ID No. 129) | 465 | 17.9 | 77.0 |
| | R......R (SEQ ID No. 130) | 402 | 15.5 | 92.5 |
| | K..NN..R (SEQ ID No. 131) | 92 | 3.5 | 96.1 |
| | R..N.... (SEQ ID No. 132) | 13 | 0.5 | 96.6 |
| | K...N..R (SEQ ID No. 133) | 11 | 0.4 | 97.0 |
| | ......Y. (SEQ ID No. 134) | 10 | 0.4 | 97.4 |
| | ...N.... (SEQ ID No. 135) | 8 | 0.3 | 97.7 |
| | E..N.... (SEQ ID No. 136) | 7 | 0.3 | 98.0 |
| Epitope | GKCNS (SEQ ID No. 137) | 2097 | 80.8 | 80.8 |
| C2 | .N.S. (SEQ ID No. 138) | 140 | 5.4 | 86.2 |
| (n = 2594) | .T.S. (SEQ ID No. 139) | 93 | 3.6 | 89.8 |
| | DN... (SEQ ID No. 140) | 81 | 3.1 | 92.9 |
| | DT.I. (SEQ ID No. 141) | 60 | 2.3 | 95.3 |
| | .N... (SEQ ID No. 142) | 42 | 1.6 | 96.9 |
| | D.... (SEQ ID No. 143) | 24 | 0.9 | 97.8 |
| | .T.I. (SEQ ID No. 144) | 23 | 0.9 | 98.7 |
| | ...K. (SEQ ID No. 145) | 7 | 0.3 | 99.0 |
| | V.... (SEQ ID No. 146) | 5 | 0.2 | 99.2 |
| Epitope | KRSQQTVIPNIGS (SEQ ID No. 147) | 2264 | 87.3 | 87.3 |
| D | ............F (SEQ ID No. 148) | 55 | 2.1 | 89.4 |
| (n = 2594) | R.....I...... (SEQ ID No. 149) | 52 | 2.0 | 91.4 |
| | .........D..Y (SEQ ID No. 150) | 33 | 1.3 | 92.7 |
| | ......I...... (SEQ ID No. 151) | 27 | 1.0 | 93.7 |
| | .......T..... (SEQ ID No. 152) | 23 | 0.9 | 94.6 |
| | ......I...V.. (SEQ ID No. 153) | 20 | 0.8 | 95.4 |
| | ............P (SEQ ID No. 154) | 15 | 0.6 | 96.0 |
| | ..........V.. (SEQ ID No. 155) | 15 | 0.6 | 96.5 |
| | R............ (SEQ ID No. 156) | 15 | 0.6 | 97.1 |
| Epitope | ENCTLIDALLGDPQCDGFQNKK (SEQ ID No. 157) | 1285 | 49.5 | 49.5 |
| E | .............H.......E (SEQ ID No. 158) | 728 | 28.1 | 77.6 |
| (n = 2594) | K............H.......E (SEQ ID No. 159) | 275 | 10.6 | 88.2 |
| | K............H......E. (SEQ ID No. 160) | 49 | 1.9 | 90.1 |
| | ID...........H..V...ET (SEQ ID No. 161) | 44 | 1.7 | 91.8 |
| | .....................N (SEQ ID No. 162) | 39 | 1.5 | 93.3 |
| | .............H........ (SEQ ID No. 163) | 29 | 1.1 | 94.4 |
| | ID...........H......ET (SEQ ID No. 164) | 23 | 0.9 | 95.3 |
| | I............H......E. (SEQ ID No. 165) | 21 | 0.8 | 96.1 |
| | .............H..S....E (SEQ ID No. 166) | 9 | 0.3 | 96.5 |

The present invention will be further clarified by the following examples, which are intended to be exemplary of the present invention.

### EXAMPLES

### Example 1

Collection of the influenza A HA sequences and extraction of HA1 region as well as the epitope regions recognized by two independent HuMAbs: B-1 and D-1. Full-length protein sequences of HA of human-, swine- and avian-derived influenza A viruses H1N1 including H1N1 pdm, H3N2 and H5N1 were obtained from the Influenza Virus Resource at the National Center for Biotechnology Information (http://www.ncbi.nlm.nih.gov/genomes/FLU/FLU.html) (Bao et al., 2008). The HA sequences were then aligned using mafft v6.240 (Katoh, K., Toh, H., 2008, “Recent developments in the MAFFT multiple sequence alignment program,” Brief. Bioinform. 9, 286-298). To determine HA1 regions, palindrome sequence identified just after the cleavage point (n′-GLFGAIAGFI-c′ were located, the palindrome region is underlined) (Skehel, J. J., Waterfield, M. D., 1975, “Studies on the primary structure of the influenza virus hemagglutinin,” Proc. Nat. Acad. Sci. USA 72, 93-97).

Two HuMAbs (B-1 and D-1) were previously prepared, independently from two vaccinated volunteers, that showed strong neutralizing activities against global strains of influenza A virus H3N2. The epitope regions were identified using a total of 158 sets of 15-residue peptides (overlapping by 13 amino acids) spanning aa 1 to 329 of HA1 region of human H3N2: 173-181 (SEQ ID NO: 2) according to the positive reactions with 4 peptides (aa 167-181, aa 169-183, aa 171-185 and aa 173-187) (SEQ ID NOs: 167-170) and aa 227-239 (SEQ ID NO: 3) according to the positive reactions with 2 peptides (aa 225-239 and aa 227-241) (SEQ ID NOs: 171, 172) (Kubota-Koketsu et al.). The sequence of aa 167-187 at a position from N-terminus in HA1 region of human H3N2 of the influenza A virus corresponds an “upper part” of human H3N2 of the influenza A virus and aa 225-241 at a position from N-terminus in HA1 region of human H3N2 of the influenza A virus corresponds a “lower part” of human H3N2 of the influenza A virus. It should be understood that various subtypes of influenza A virus comprise amino acid sequences corresponding to the “upper part,” “lower part,” “upper region,” and “lower region” of human H3N2. Sequences corresponding to the upper and the lower regions of human H3N2 were also extracted from virus strains of different host origin or from those belonging to other influenza A virus subtypes from the Influenza Virus Resource at the National Center for Biotechnology Information (Bao et al., 2008): H1N1 derived from human and swine, and H1N1 pdm from human; H3N2 derived from human and swine; and H5N1 derived from human and avian.

The known neutralizing epitopes A to E (Webster and Laver, 1980; Wiley et al., 1981) were similarly extracted as control sequences from the human H3N2 sequences of the Influenza Virus Resource at the National Center for Biotechnology Information (Bao et al., 2008).

Sequence comparison Sequence logos for the epitope sequences recognized by B-1 and D-1 and the corresponding sequences in the human-, swine- and avian-derived viruses were constructed using weblogo (Crooks, G. E., Hon, G., Chandonia, J. M., Brenner, S. E., 2004, “WebLogo: A sequence logo generator,” Genome Res. 14, 1188-1190).

The evolutionary relationship was inferred using the Minimum Evolution (ME) method (Rzhetsky, A., Nei, M., 1992, “A simple method for estimating and testing minimum evolution trees,” Mol. Biol. Evol. 9, 945-967). The tree is drawn to scale, with branch lengths in the same units as those of the evolutionary distances used to infer the phylogenetic tree. The evolutionary distances were computed using the Poisson correction method (Zuckerkandl, E., Pauling, L., 1965, “Evolutionary divergence and convergence in proteins. In Evolving Genes and Proteins,” (V. Bryson, H. J. Vogel, Ed.), pp. 97-166, Academic Press, New York) and are in the units of the number of amino acid substitutions per site. The ME tree was searched using the Close-Neighbor-Interchange algorithm (Nei, M., Kumar, S., 2000, “Molecular Evolution and Phylogenetics,” Oxford University Press, New York.) at a search level of 1. The Neighbor-joining algorithm (Saitou, N., Nei, M., 1987, “The neighbor-joining method: A new method for reconstructing phylogenetic trees,” Mol. Biol. Evol. 4, 406-425) was used to generate the initial tree. All positions containing gaps and missing data were eliminated from the dataset (Complete deletion option). Phylogenetic analyses were conducted in MEGA4 (Tamura, K., Dudley, J., Nei, M., Kumar, S., 2007, “MEGA4: Molecular Evolutionary Genetics Analysis (MEGA) software version 4.0,” Mol. Biol. Evol. 24, 1596-1599).

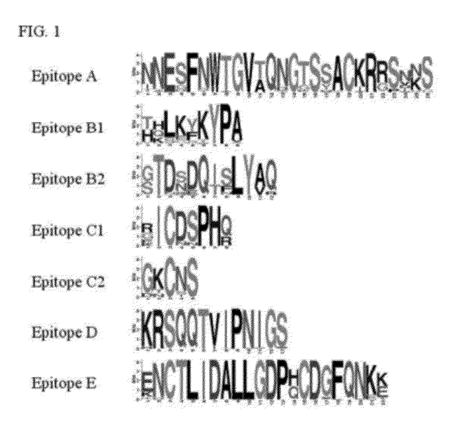

FIG. 1 shows the sequence logo of known neutralizing epitopes A-E. The sequences of the known neutralizing epitopes A-E in human H3N2, extracted from the Influenza Virus Resource at the National Center for Biotechnology Information, were subjected to sequence logo for analysis of variation sites and their ratios. The sequence logo analysis using all these variants (Table 1) in individual epitope regions also supported the high heterogeneity of these known neutralizing epitopes, especially in epitopes A and B2, as well as B1 (FIG. 1).

The possible detection of the corresponding beta-sheet regions in HA molecules was examined in other subtypes of influenza A virus. A total of 4301, 283, and 1534 full-length HA sequences derived from human (H1N1, H1N1 pdm, H3N2 and H5N1), swine (H1N1 and H3N2), and avian (H5N1) influenza A virus were obtained from the Influenza Virus Resource at the National Center for Biotechnology Information (Bao et al., 2008), respectively, at May 15, 2009, except for H1N1 pdm at Jun. 12, 2009. Sequences corresponding to the upper and the lower regions of H3N2 were also extracted from full-length HA sequences of H1N1 and H5N1 derived from different hosts. The efficiency to produce variants was much higher in swine—than human-derived influenza viruses, H1N1 and H3N2 (Table 1). Especially, H1N1 pdm showed perfect conservation among the 312 isolates, except for one variant in the lower region (Table 1).

The top ten variants and percentages of individual sequences in the upper and the lower regions are shown in Table 3 below. It should be noted that individual sequences in the upper and the lower regions of H3N2 are shown in Table 3, as well as sequences “corresponding” to the upper and the lower regions of H3N2 in other influenza A virus subtypes.

TABLE 3
Amino acid variation of the upper and lower regions in individual subtypes.

| | | | | Cumu- | | | | Cumu- |
|---|---|---|---|---|---|---|---|---|
| | | | Appear- | lative | | | Appear- | lative |
| | Upper | Sequence | ance | ratio | Lower | Sequence | ance | ratio |
| Virus | sequence | number | ratio (%) | (%) | sequence | number | ratio (%) | (%) |
| Human | EKEVLVLWG | 1090 | 93.1 | 93.1 | EGRINYYWTLLEP | 1097 | 93.7 | 93.7 |
| H1N1 | (SEQ ID No. 34) | | | | (SEQ ID No. 51) | | | |
| (n = 1171) | G........ | 59 | 5.0 | 98.1 | A............ | 21 | 1.8 | 95.5 |
| | (SEQ ID No. 35) | | | | (SEQ ID No. 52) | | | |
| | K........ | 8 | 0.7 | 98.8 | A..M......... | 12 | 1.0 | 96.5 |
| | (SEQ ID No. 36) | | | | (SEQ ID No. 53) | | | |
| | ......M.. | 6 | 0.5 | 99.3 | A..M.......K. | 10 | 0.9 | 97.4 |
| | (SEQ ID No. 37) | | | | (SEQ ID No. 54) | | | |
| | .....I... | 2 | 0.2 | 99.5 | H..M......... | 5 | 0.4 | 97.8 |
| | (SEQ ID No. 38) | | | | (SEQ ID No. 55) | | | |
| | K.....I.. | 1 | 0.1 | 99.6 | A..M......V.. | 5 | 0.4 | 98.2 |
| | (SEQ ID No. 39) | | | | (SEQ ID No. 56) | | | |
| | .....L... | 1 | 0.1 | 99.7 | P..M......... | 4 | 0.3 | 98.5 |
| | (SEQ ID No. 40) | | | | (SEQ ID No. 57) | | | |
| | D........ | 1 | 0.1 | 99.7 | ....S........ | 4 | 0.3 | 98.9 |
| | (SEQ ID No. 41) | | | | (SEQ ID No. 58) | | | |
| | ..K...... | 1 | 0.1 | 99.8 | ......H...... | 2 | 0.2 | 99.1 |
| | (SEQ ID No. 42) | | | | (SEQ ID No. 59) | | | |
| | ...I..... | 1 | 0.1 | 99.9 | A..M......I.. | 2 | 0.2 | 99.2 |
| | (SEQ ID No. 43) | | | | (SEQ ID No. 60) | | | |
| Swine | GKEVLVLWG | 104 | 55.3 | 55.3 | AGRMNYYWTLIEP | 93 | 49.5 | 49.5 |
| H1N1 | (SEQ ID No. 35) | | | | (SEQ ID No. 60) | | | |
| (n = 188) | ......I.. | 29 | 15.4 | 70.7 | ..........LDQ | 36 | 19.1 | 68.6 |
| | (SEQ ID No. 44) | | | | (SEQ ID No. 61) | | | |
| | E........ | 23 | 12.2 | 83.0 | ..........V.. | 23 | 12.2 | 80.9 |
| | (SEQ ID No. 34) | | | | (SEQ ID No. 62) | | | |
| | K........ | 10 | 5.3 | 88.3 | ..........L.. | 7 | 3.7 | 84.6 |
| | (SEQ ID No. 36) | | | | (SEQ ID No. 63) | | | |
| | R.....I.. | 6 | 3.2 | 91.5 | ......H...... | 5 | 2.7 | 87.2 |
| | (SEQ ID No. 46) | | | | (SEQ ID No. 64) | | | |
| | K.....I.. | 4 | 2.1 | 93.6 | T.........V.. | 5 | 2.7 | 89.9 |
| | (SEQ ID No. 39) | | | | (SEQ ID No. 65) | | | |
| | .....II.. | 3 | 1.6 | 95.2 | ....D.....V.. | 5 | 2.7 | 92.6 |
| | (SEQ ID No. 47) | | | | (SEQ ID No. 66) | | | |
| | .R....... | 2 | 1.1 | 96.3 | ...I......LDQ | 2 | 1.1 | 93.6 |
| | (SEQ ID NO. 48) | | | | (SEQ ID No. 67) | | | |
| | ..K...... | 2 | 1.1 | 97.3 | E..I......L.. | 2 | 1.1 | 94.7 |
| | (SEQ ID No. 49) | | | | (SEQ ID No. 51) | | | |
| | E.......A | 1 | 0.5 | 97.9 | T.........L.. | 1 | 0.5 | 95.2 |
| | (SEQ ID No. 50) | | | | (SEQ ID No. 68) | | | |
| H1N1 | GKEVLVLWG | 312 | 100.0 | 100.0 | EGRMNYYWTLVEP | 310 | 99.4 | 99.4 |
| pdm | (SEQ ID No. 35) | | | | (SEQ ID No. 69) | | | |
| (n = 312) | | | | | ..........I.. | 2 | 0.6 | 100.0 |
| | | | | | (SEQ ID No. 70) | | | |
| Human | KFDKLYIWG | 2115 | 81.5 | 81.5 | SSRISIYWTIVKP | 1526 | 58.8 | 58.8 |
| H3N2 | (SEQ ID No. 2) | | | | (SEQ ID No. 21) | | | |
| (n = 2594) | E........ | 160 | 6.2 | 87.7 | P............ | 969 | 37.4 | 96.2 |
| | (SEQ ID No. 8) | | | | (SEQ ID No. 3) | | | |
| | N........ | 157 | 6.1 | 93.8 | ..I.......... | 24 | 0.9 | 97.1 |
| | (SEQ ID No. 9) | | | | (SEQ ID No. 22) | | | |
| | Q........ | 123 | 4.7 | 98.5 | ..K.......... | 16 | 0.6 | 97.7 |
| | (SEQ ID No. 10) | | | | (SEQ ID No. 23) | | | |
| | NS....... | 15 | 0.6 | 99.1 | ...V......... | 12 | 0.5 | 98.2 |
| | (SEQ ID No. 11) | | | | (SEQ ID No. 24) | | | |
| | R........ | 6 | 0.2 | 99.3 | ..G.......... | 10 | 0.4 | 98.6 |
| | (SEQ ID No. 12) | | | | (SEQ ID No. 25) | | | |
| | ......V.. | 5 | 0.2 | 99.5 | ..X.......... | 6 | 0.2 | 98.8 |
| | (SEQ ID No. 13) | | | | (SEQ ID No. 26) | | | |
| | ..E...... | 4 | 0.2 | 99.7 | ......H...... | 4 | 0.2 | 99.0 |
| | (SEQ ID No. 14) | | | | (SEQ ID No. 27) | | | |
| | .Y....... | 2 | 0.1 | 99.7 | ............S | 3 | 0.1 | 99.1 |
| | (SEQ ID No. 15) | | | | (SEQ ID No. 28) | | | |
| | G........ | 1 | 0.0 | 99.8 | ....N........ | 2 | 0.1 | 99.2 |
| | (SEQ ID No. 16) | | | | (SEQ ID No. 29) | | | |
| Swine | KFDKLYIWG | 47 | 49.5 | 49.5 | SSRISIYWTIVKP | 66 | 69.5 | 69.5 |
| H3N2 | (SEQ ID No. 2) | | | | (SEQ ID No. 21) | | | |
| (n = 95) | D........ | 19 | 20.0 | 69.5 | ..I.......... | 16 | 16.8 | 86.3 |
| | (SEQ ID No. 17) | | | | (SEQ ID No. 22) | | | |
| | NS....... | 7 | 7.4 | 76.8 | ...V......... | 7 | 7.4 | 93.7 |
| | (SEQ ID No. 11) | | | | (SEQ ID No. 24) | | | |
| | N........ | 6 | 6.3 | 83.2 | ............S | 2 | 2.1 | 95.8 |
| | (SEQ ID No. 9) | | | | (SEQ ID No. 28) | | | |
| | N.....V.. | 6 | 6.3 | 89.5 | PG........... | 1 | 1.1 | 96.8 |
| | (SEQ ID No. 18) | | | | (SEQ ID No. 30) | | | |
| | N.N...... | 3 | 3.2 | 92.6 | ...M......... | 1 | 1.1 | 97.9 |
| | (SEQ ID No. 19) | | | | (SEQ ID No. 31) | | | |
| | D.N...... | 3 | 3.2 | 95.8 | .........T... | 1 | 1.1 | 98.9 |
| | (SEQ ID No. 20) | | | | (SEQ ID No. 32) | | | |
| | E........ | 1 | 1.1 | 96.8 | ....G.H...... | 1 | 1.1 | 100.0 |
| | (SEQ ID No. 8) | | | | (SEQ ID No. 33) | | | |
| | G........ | 1 | 1.1 | 97.9 | | | | |
| | (SEQ ID NO. 16) | | | | | | | |
| | ......V.. | 1 | 1.1 | 98.9 | | | | |
| | (SEQ ID No. 13) | | | | | | | |
| Human | QEDLLVLWG | 195 | 87.1 | 87.1 | SGRMEFFWTILKP | 166 | 74.1 | 74.1 |
| H5N1 | (SEQ ID No. 71) | | | | (SEQ ID No. 82) | | | |
| (n = 224) | ......M.. | 13 | 5.8 | 92.9 | ...........N. | 14 | 6.2 | 80.4 |
| | (SEQ ID No. 72) | | | | (SEQ ID No. 83) | | | |
| | .....I... | 12 | 5.4 | 98.2 | ....D........ | 12 | 5.4 | 85.7 |
| | (SEQ ID No. 73) | | | | (SEQ ID No. 84) | | | |
| | ..N..I... | 2 | 0.9 | 99.1 | ............S | 11 | 4.9 | 90.6 |
| | (SEQ ID No. 74) | | | | (SEQ ID No. 85) | | | |
| | ......I.. | 1 | 0.4 | 99.6 | .......A.... | 8 | 3.6 | 94.2 |
| | (SEQ ID No. 75) | | | | (SEQ ID No. 86) | | | |
| | R........ | 1 | 0.4 | 100.0 | N............ | 4 | 1.8 | 96.0 |
| | (SEQ ID No. 76) | | | | (SEQ ID No. 87) | | | |
| | | | | | ...I........S | 3 | 1.3 | 97.3 |
| | | | | | (SEQ ID No. 88) | | | |
| | | | | | ...I......... | 3 | 1.3 | 98.7 |
| | | | | | (SEQ ID No. 89) | | | |
| | | | | | ....D....M.R. | 1 | 0.4 | 99.1 |
| | | | | | (SEQ ID No. 90) | | | |
| | | | | | .......R..... | 1 | 0.4 | 99.6 |
| | | | | | (SEQ ID No. 91) | | | |
| Avian | QEDLLVLWG | 1183 | 77.1 | 77.1 | SGRMEFFWTILKP | 1059 | 69.0 | 69.0 |
| H5N1 | (SEQ ID No. 71) | | | | (SEQ ID No. 82) | | | |
| (n = 1534) | .....I... | 194 | 12.6 | 89.8 | ....D........ | 239 | 15.6 | 84.6 |
| | (SEQ ID No. 73) | | | | (SEQ ID No. 84) | | | |
| | ......M.. | 111 | 7.2 | 97.0 | ............S | 78 | 5.1 | 89.7 |
| | (SEQ ID No. 72) | | | | (SEQ ID No. 85) | | | |
| | V....II.. | 8 | 0.5 | 97.5 | ...ID........ | 30 | 2.0 | 91.7 |
| | (SEQ ID No. 77) | | | | (SEQ ID No. 92) | | | |
| | ......I.. | 5 | 0.3 | 97.8 | ...V........S | 26 | 1.7 | 93.4 |
| | (SEQ ID No. 75) | | | | (SEQ ID No. 93) | | | |
| | ..N..I... | 5 | 0.3 | 98.2 | ...I........S | 26 | 1.7 | 95.0 |
| | (SEQ ID No. 74) | | | | (SEQ ID No. 88) | | | |
| | K....I... | 4 | 0.3 | 98.4 | ...I......... | 20 | 1.3 | 96.3 |
| | (SEQ ID No. 78) | | | | (SEQ ID No. 89) | | | |
| | P........ | 3 | 0.2 | 98.6 | ....D....M... | 10 | 0.7 | 97.0 |
| | (SEQ ID No. 79) | | | | (SEQ ID No. 94) | | | |
| | .....IM.. | 2 | 0.1 | 98.8 | ...........R. | 6 | 0.4 | 97.4 |
| | (SEQ ID No. 80) | | | | (SEQ ID No. 95) | | | |
| | M....I... | 2 | 0.1 | 98.9 | ...........E. | 5 | 0.3 | 97.7 |
| | (SEQ ID No. 81) | | | | (SEQ ID No. 96) | | | |

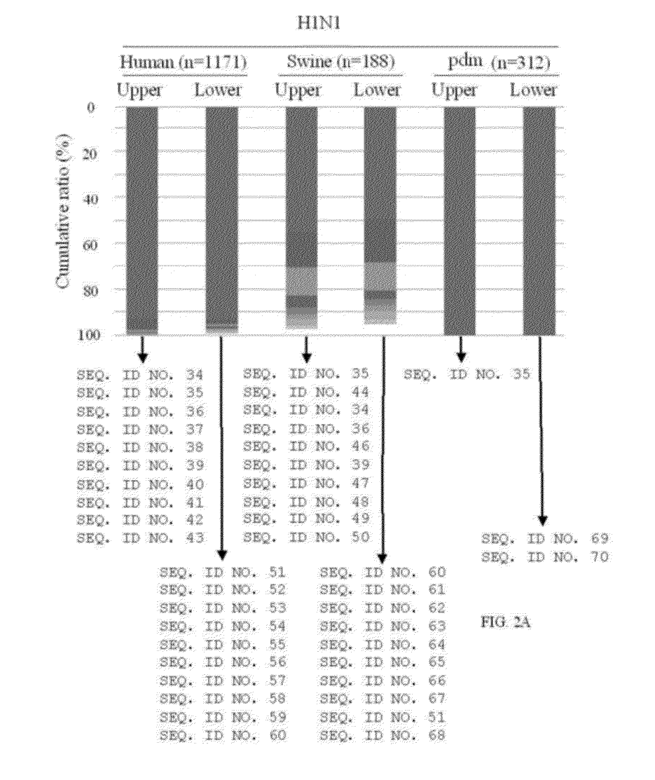

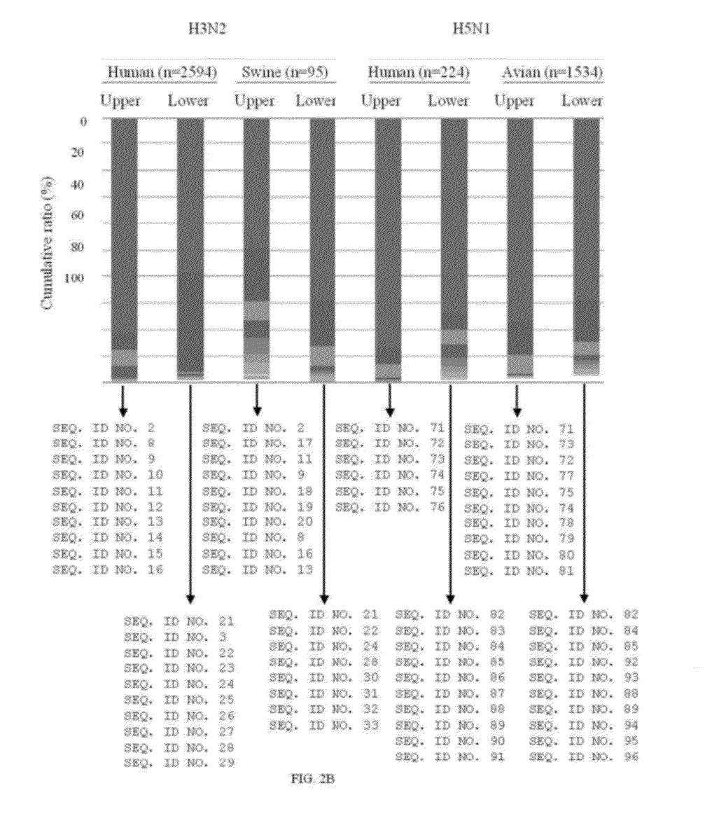

The data in Table 3 was summarized and illustratively shown for the individual variant ratios in FIGS. 2A and 2B. FIGS. 2A and 2B show the sequences and the sequence variation and accumulated rates at the upper and the lower sequences. The accumulated rates of top 10 sequences at the upper and the lower regions in H1N1 derived from human and swine as well as H1N1 pdm; H3N2 derived from human and swine; and H5N1 derived from human and avian, as shown in Table 3, are shown in FIGS. 2A and 2B. The sequence of the highest rate was used as consensus and next rate sequences were serially laid out. In the Tables of the present application, the period (.) used in the next rate sequences indicates the same amino acid residue. The sequences in human H1N1 and H1N1 pdm were highly conserved in both the upper and the lower regions. In case of human and avian H5N 1, the sequences of both regions were relatively highly conserved. The lower region of human H3N2 was fairly conserved and the upper region of this subtype was more highly conserved than the lower peptide.

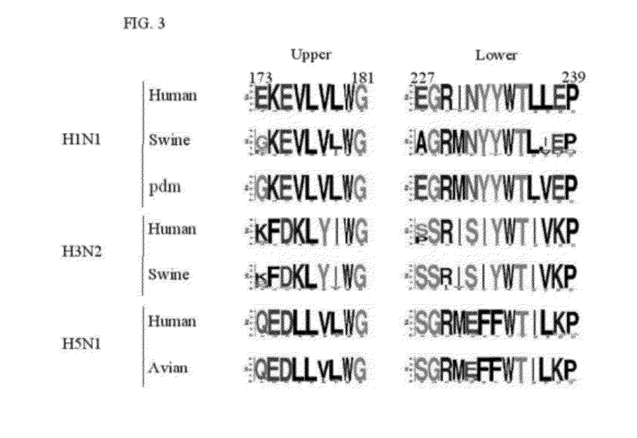

Sequence logo analysis shown in FIG. 3 using all the variants (Table 1) supported this conclusion. FIG. 3 shows the sequence logo of the upper and the lower regions of various subtypes of influenza A virus. The upper and the lower regions were extracted from the Influenza Virus Resource at the National Center for Biotechnology Information: human H1N1 (n=1171), swine H1N1 (n=188), and H1N1 pdm (n=312); human H3N2 (n=2594) and swine H3N2 (n=95); and human H5N1 (n=224) and avian H5N1 (n=1534). All of the variants in individual subtypes with different host origin were used for this sequence logo analysis. Amino acid residues by H3 numbering are shown at the top of the sequence. Appearance of variants was mainly due to the variations in the restricted amino acid residues in most cases: residues 173 and 227 in human H3N2; residues 173, 179, 229 and 230 in swine H3N2; residues 173, 179, 237, 238 and 239 in swine H1N1; and residues 178, 179, 231 and 239 in human and avian H5N 1. Consequently, the upper and the lower regions were highly conserved in human H1N1 and H1N1 pdm.

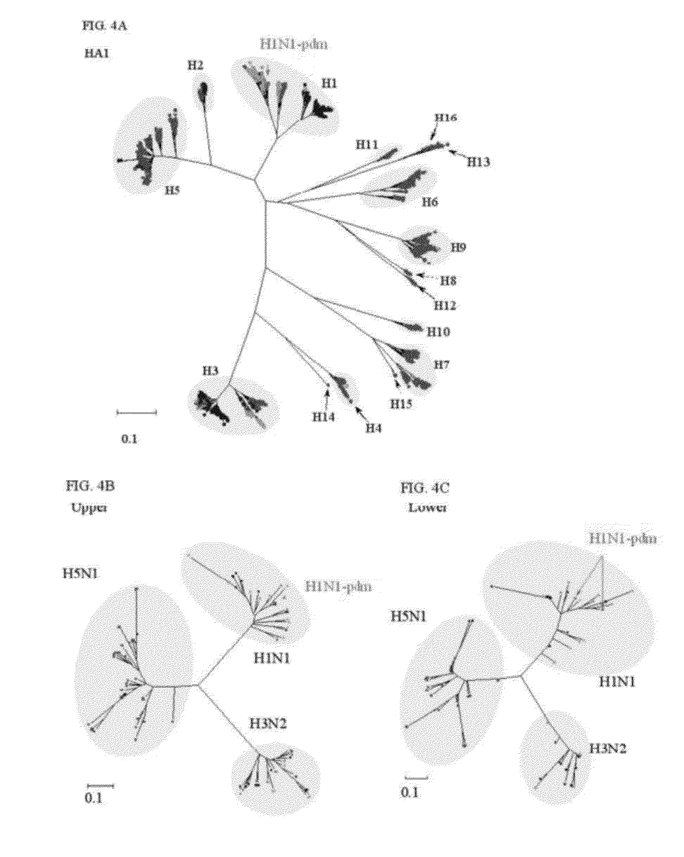

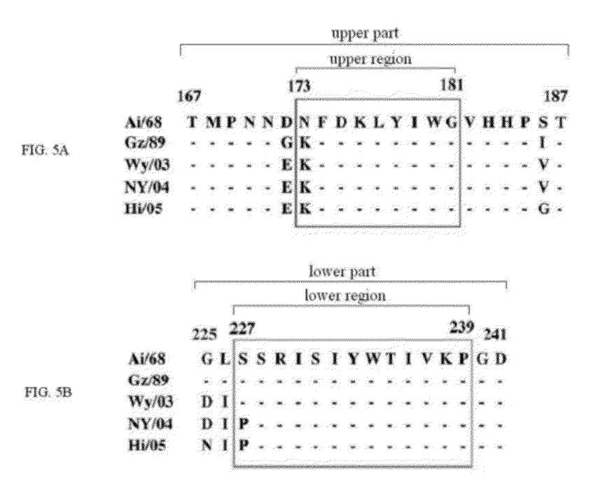

Phylogenetic tree analysis of the regions derived from virus strains of different hosts revealed their independent clustering among H1N1, H3N2 and H5N1 subtypes (FIG. 4). Thus, the H3N2 regions recognized by neutralizing HuMAbs as well as the corresponding regions in HIN1 and H5N1 seem to evolve independently.

There was no apparent difference in the conservation of both regions in H5N 1 of human- and avian-origins, indicating the transmission of several variants produced in avian to human. The 312 sequences of the upper and the lower regions in H1N1 pdm were completely the same, except for two sequences in the upper region. This result is reasonable if only a single strain of swine-origin influenza A H1N1 has acquired the ability to transmit to human.

FIGS. 5A-5B show the antigenic regions of broadly neutralizing HuMAbs based on the reactivity of B-1 and D-1 HuMAbs with a series of overlapping peptides covering sequences of HA1 region, amino acid sequences from aa 167-187 and from aa 225-241 (SEQ ID NOs: 4 and 5). Both MAbs showed the same responses to the synthesized peptides, i.e., significant responses to 2 regions by 4 (aa 167-181, aa 169-183, aa 171-185 and aa 173-187) (SEQ ID NOs: 167-170) and 2 (aa 225-239 and aa 227-241) (SEQ ID NOs: 171-172) peptides, spanning the overlapping regions aa 173-181 (SEQ ID NO: 2) and 227-239 (SEQ ID NO: 3), respectively. Both HuMAbs recognized two separate regions (aa 173-181 (SEQ ID NO: 2), and aa 227-239 (SEQ ID NO: 3)), located close to each other in the three dimensional structure of HA1, a novel human epitope. This result suggested that B-1 and D-1 recognized a conformational epitope.

### EXAMPLE 2

Neutralization test with culture supernatant of human PBMCs stimulated with synthetic peptides. A total of 4 healthy volunteers were selected for obtaining PBMCs to test neutralization with culture supernatant of human PBMCs stimulated with synthetic peptides, SEQ ID NO: 4 (upper part comprising the upper region) and SEQ ID NO: 5 (lower part comprising the lower region). Ten or twenty milliliters of blood were obtained from individual volunteers. The PBMCs were prepared by centrifugation through Ficoll Pack Plus (GE Healthcare, Uppsala, Sweden) for 40 min at 520×g. The cells were washed with serum free RPMI1640 culture medium and suspended in RPMI1640 medium supplemented with 10% fetal calf serum. The cells were incubated for 1 day at 37 degrees C. in a 5% CO₂ atmosphere. The cells were suspended at concentration of 2×10⁶ cells/mL in RPMI1640 medium supplemented with 10% fetal calf serum and were added the mitogen PWM (5 microgram per milliliter). The cells were incubated for 1 day at 37 degrees C. in a 5% CO₂ atmosphere. The stimulated cells were washed with serum free RPMI1640 culture medium and were suspended in RPMI1640 medium supplemented with 10% fetal calf serum and were added the synthetic peptide (10 microgram per milliliter) or mixed peptides (5 microgram per milliliter respectively). The cells were incubated for 7 days at 37 degrees C. in a 5% CO₂ atmosphere. The culture supernatants of stimulated cells were collected and diluted with PBS(-) to 1:10 after treated with RDE(II) (Denka seiken, Japan). The diluted culture supernatants were used for micro viral neutralization (VN) test (Y. Okuno, K. Tanaka, K. Baba, A. Maeda, N. Kunita, S. Ueda, Rapid focus reduction neutralization test of influenza A and B viruses in microtiter system, J. Clin. Microbiol. 28 (1990) 1308-1313). The virus was A/Hiroshima/52/2005(H3N2) strain. The VN activity was indicated by focus reduction rate (%) and by N ratio. N ratio is equivalent to the VN activity of culture supernatant of PBMCs stimulated with synthetic peptides/the activity of culture supernatant of PBMCs stimulated without synthetic peptides.

### EXAMPLE 3

Evaluation of epitope peptide antigenicity in mouse Immunization of mouse Peptides corresponding to the upper region and the lower region highly conserved in HA of human H1N1, H3N2 and H5N1, were synthesized as described below.

(a) H1N1, upper peptide: CKEVLVLWG (SEQ ID NO: 177), cysteine is added at the N-terminus of the sequence which lacks one amino acid at the N-terminus of SEQ ID NO: 34.
(b) H1N1, lower peptide: CGRINYYWTLLEP (SEQ ID NO:178), cysteine is added at the N-terminus of the sequence which lacks one amino acid at the N-terminus of SEQ ID NO: 51.
(c) H3N2, upper peptide: CFDKLYIWG (SEQ ID NO: 179), cysteine is added at the N-terminus of aa 174-181 of SEQ ID NO:1 (the sequence of aa 174-181 lacks one amino acid at the N-terminus of SEQ ID NO: 2).
(d) H3N2, lower peptide: CSRISIYWTIVKP (SEQ ID NO: 180), cysteine is added at the N-terminus of aa 228-239 of SEQ ID NO: 1 (the sequence of aa 228-239 lacks one amino acid at the N-terminus of SEQ ID NO: 3)
(e) H5N1, upper peptide: QEDLLVLWGC (SEQ ID NO: 181), cysteine is added at the C-terminus of aa 173-181 (SEQ ID NO: 71). (f) H5N1, lower peptide: SGRMEFFWTILKPC (SEQ ID NO: 182), cysteine is added at the C-terminus of aa 227-239 (SEQ ID NO: 82).
Cysteine was added for conjugating a carrier to the peptide. These peptides were conjugated with KLH and mixed as an immunogen. Suspension of twenty-five microliter of mixture of the peptides and complete Freund's adjuvant (1:1) was injected into the mouse at 1, 7, and 14 days. One week after the final injection, blood was collected from the mouse.

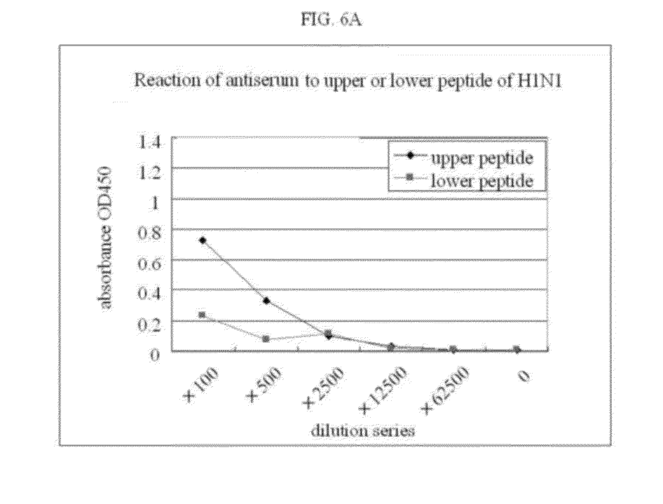

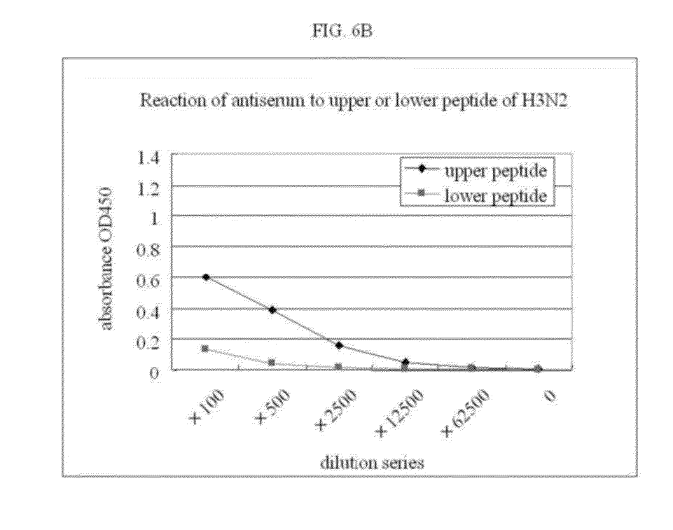

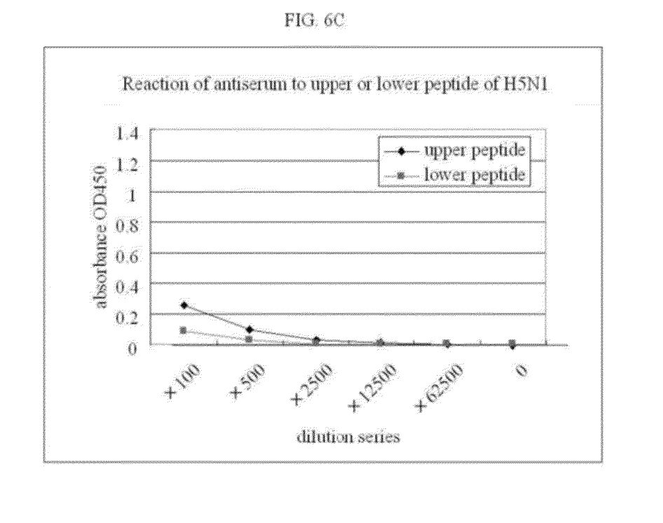

ELISA (Enzyme-linked immunosorbent assay). Microtiter plates were coated with 1 microgram per milliliter of peptide in PBS at 4 degrees C. overnight and were blocked with PBS containing 5% BSA at 4 degrees C. overnight. After discarding blocking solution, serum was diluted to several concentrations in PBS and added to each well for 1hr at room temperature. Then plates were washed 3 times with 0.05% Tween20-PBS and HRP conjugated goat anti-mouse IgG (MBL code 330) was added to each well and reacted for l hr at room temperature. After washing 3 times with 0.05% Tween20-PBS, substrate reagent was added to each well. The reaction was stopped with 2N H₂SO₄. Finally, the absorbance was measured. FIGS. 6A-6C show the reaction of antiserum to the upper peptide or the lower peptide of H1N1, H3N2, and H5N 1. As a result, it was confirmed that each serum reacted to the upper peptide more strongly than the lower peptide.

### EXAMPLE 4

Evaluation of Epitope Peptide Antigenicity in Rabbit Immunization of Rabbit

The upper part peptide (TMPNNEKFDKLYIWGVHHPGT) (SEQ ID NO: 4) or the lower part peptide (NIPSRISIYWTIVKPGD) (SEQ ID NO. 5) was conjugated with KLH, or synthesized as 8 branched MAP (Multiple antigen peptide) form. New Zealand white rabbits were immunized subcutaneously as follows: 1) immunized by cocktail with the upper and the lower part peptides which were conjugated with KLH; 2) immunized by cocktail with MAP forms of the upper and the lower part peptides; and 3) immunized with KLH conjugated the upper or the lower part peptide. For each protocol, two rabbits were immunized. For first immunization (1 mg peptide per rabbit), antigen was administered with Freund's complete adjuvant , thereafter for 1 month, boost immunization(0.5 mg peptide per rabbit) was carried out with Freund's incomplete adjuvant 3 times once in every 2 weeks. The blood was collected 1 week after final immunization and anti-serum was prepared.

### ELISA for Detecting Anti-Epitope Peptide Antibody.

N-terminal biotinylated epitope peptide, which had been diluted to 1 microgram per milliliter in SuperBlock T20 Blocking Buffer (Thermo), was added to a well (50 microliter) of 96-well streptavidin pre-coated microplate plate (Nunc). The microplate was incubated for 30 minutes at room temperature, and then the biotinylated peptide solution was discarded. A solution containing 3% Skim Milk in PBS(-) was added thereto at room temperature over the period of at least 1 hour. The microplate was washed with water 3 times. The anti-serum diluted in a solution containing 3% skim Milk in PBS(-) was added to a well and incubated for 1.5 hour at room temperature. The microplate was washed with PBS(-) containing 0.05% Tween20 and 50 uL of peroxidase labeled anti rabbit IgG (CELL LAB) which was diluted to 4000 folds with Superblock T20 was added to a well and incubated for 1 hour at room temperature. After the microplate was washed with a PBS(-) containing 0.05% Tween20, 100 microliter of peroxidase substrate solution(SUMILON) was added to a well and incubated for 20 minutes at room temperature. Thereafter, 100 microliter of reaction stop solution (SUMILON) was added to a well and the absorbance at 490 nm was assayed using a plate reader. The absorbance for anti-serum was subtracted with that for pre-immune serum to determine the specific reactivity.

### ELISA for Detecting Anti HA Antibody

HA vaccine containing A/Brisbene/59/2007(H1N1), A/Uruguay/716/2007(H3N2) and B/Florida/4/2006 was dissolved in PBS(-) (each >30 microgram per milliliter), and diluted to 30 folds with PBS(-). Diluted HA vaccine solution was added to a well (50 microliter) of 96 well microplate (Maxisorp, Nunc). The microplate was incubated at 5 degrees C. overnight and then the HA solution was discarded. A solution containing 3% Skim Milk in PBS(-) was added thereto at room temperature over the period of at least 1 hour. The microplate was washed with water 3 times. The anti-serum diluted in a solution containing 3% skim Milk in PBS(-) was added to a well and incubated for 1.5 hours at room temperature. The microplate was washed with PBS(-) containing 0.05% Tween20 and 50 microliter of peroxidase labeled anti rabbit IgG (CELL LAB), which was diluted to 4000 folds with Superblock T20, was added to a well and incubated for 1 hour at room temperature. After the microplate was washed with a PBS(-) containing 0.05% Tween20, 100 microliter of peroxidase substrate solution (SUMILON) was added to each well and incubated for 20 minutes at room temperature. Thereafter, 100 microliter of reaction stop solution (SUMILON) was added to a well and the absorbance at 490 nm was assayed using a plate reader. The absorbance for anti-serum was subtracted with that for pre-immune serum to determine the specific reactivity.

### Result of Antibody Detection.

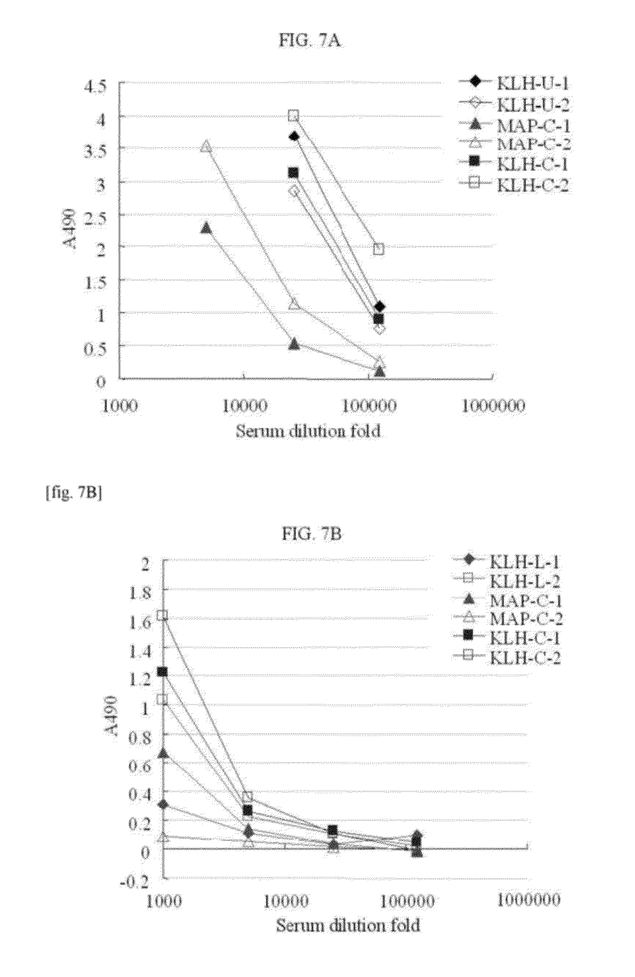

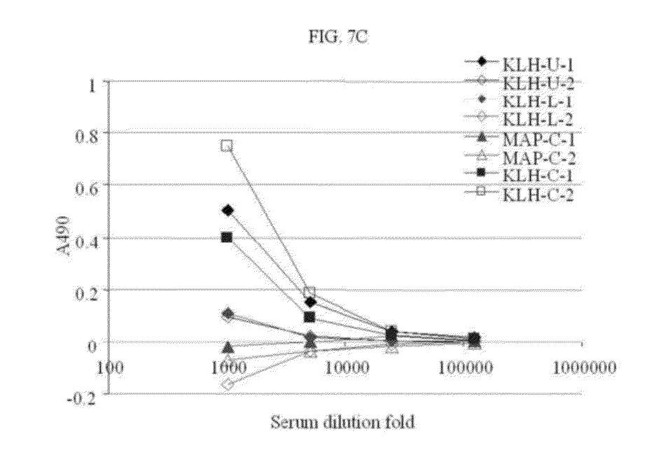

As shown in FIG. 7A and FIG. 7B, both the upper and the lower region peptides could elicit the antibody in rabbit. The upper region peptide showed high antigenicity compared with the lower region peptide. Antibody elicited by cocktail immunization with KLH-peptide conjugate and with KLH conjugated the upper region peptide reacted to HA definitely, as shown in FIG. 7C. This result indicated the possibility that immunization with epitope peptide can elicit HA reactive antibody. The abbreviations used in FIGS. 7A-7C should be understood as follows: KLH-U-1: KLH-upper part peptide immunized rabbit 1; KLH-U-2: KLH-upper part peptide immunized rabbit 2; KLH-L-1: KLH-lower part peptide immunized rabbit 1; KLH-L-2: KLH-lower part peptide immunized rabbit 2; MAP-C-1: MAP-upper part peptide and MAP-lower part peptide cocktail immunized rabbit 1; MAP-C-2: MAP-upper part peptide and MAP-lower part peptide cocktail immunized rabbit 2; KLH-C-1: KLH-upper part peptide and KLH-lower part peptide cocktail immunized rabbit 1; KLH-C-2: KLH-upper part peptide and KLH-lower part peptide cocktail immunized rabbit 2.

### EXAMPLE 5

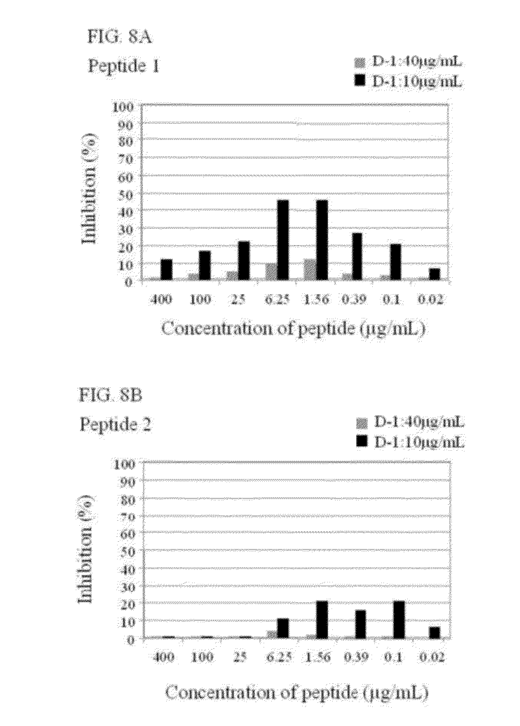

The monoclonal antibody D-1 (Kubota-Koketsu et al.) and influenza A virus were incubated with synthetic peptides (peptide 1 (SEQ ID NO: 4), and peptide 2 (SEQ ID: NO 5). The inhibition by the peptides of neutralization activity of D-1 was measured and the results are shown in FIGS. 8A-8B. The neutralization inhibition rate by peptide 1 was about 50%, and the inhibition rate by peptide 2 was about 20%. These results show that peptides 1 and 2 have reactivity with D-1 antibody. Thus, peptides 1 and 2 can induce the neutralization antibody as immunogen in human body.

### EXAMPLE 6

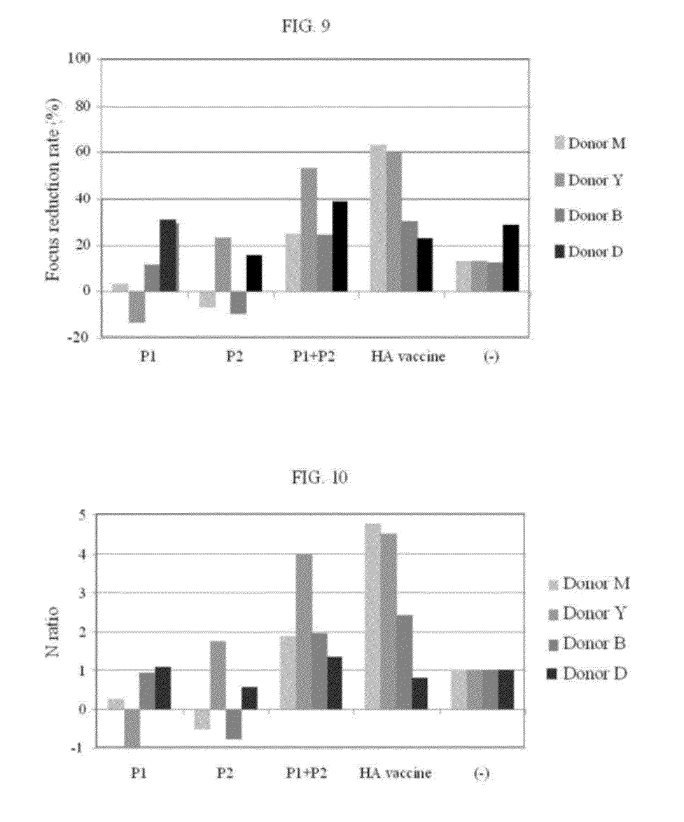

The PBMCs derived from human were incubated with synthetic peptides (peptide 1 (P1) (SEQ ID NO: 4), and peptide 2 (P2) (SEQ ID: NO 5)) The neutralization activity against influenza virus of culture supernatants was measured according to the method of Example 2 and the results are shown in FIGS. 9 and 10. HA vaccine (the purified HA vaccine antigen of A/Hiroshima/52/2005 strain (The Research Foundation for Microbial Diseases of Osaka University)) was used as positive control. FIGS. 9 and 10 showed that the stimulated PBMCs by the mixed peptides (Peptide 1+Peptide 2 (P1+P2)) had high virus neutralization activities. These results suggest that immunization with the mixed peptides induce the neutralization antibody effectively in the human body.

### INDUSTRIAL APPLICABILITY

It is expected that vaccine against influenza comprising the peptide having immunogenicity has broad spectrum over different strain belonging to the same subtype. Advantageously, there are possibilities that the vaccine has broader spectrum over different subtype. The present invention can provide the vaccine which will not be affected by the variability of the influenza virus. Also, it is expected that the antibody obtained by the selecting method of the present invention has broader cross reactivity. Thus, the present invention enables the effective treatment and/or prevention of influenza and brings the benefit in medical care.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

[Sequence Listing]

1. An antigenic peptide comprising at least an amino acid sequence of an upper region and/or a lower region of beta sheet structure within hemagglutinin HA1 region of influenza A virus.

2. The antigenic peptide according to claim 1, wherein the amino acid sequence of the upper region corresponds to amino acid residues 173-181 (SEQ ID NO: 2) within the hemagglutinin HA1 region of influenza A virus H3N2 and the amino acid sequence of the lower region corresponds to amino acid residues 227-239 (SEQ ID NO: 3) within the hemagglutinin HA1 region of influenza A virus H3N2.

3. The antigenic peptide according to claim 1, wherein the amino acid sequence of the upper region is selected from the group consisting of: SEQ ID NO: 34 (EKEVLVLWG), SEQ ID NO: 2 (KFDKLYIWG), SEQ ID NO: 71(QEDLLVLWG), and the amino acid sequence modified by substitution, insertion, addition and/or deletion of one or two amino acid (s) of SEQ IN NO: 34, 2, or 71; and wherein the amino acid sequence of the lower region is selected from the group consisting of: SEQ ID NO: 51 (EGRINYYWTLLEP), SEQ ID NO: 3 (PSRISIYWTIVKP), SEQ ID NO: 82 (SGRMEFFWTILKP), and the amino acid sequence modified by substitution, insertion, addition and/or deletion of one or two amino acid (s) of SEQ IN NO: 51, 3, or 82.

4. The antigenic peptide according to claim 1, wherein the amino acid sequence of the upper region and/or the lower region is conserved in all influenza A virus subtypes derived from human, swine, or avian hosts.

5. The antigenic peptide according to claim 1, wherein the peptide comprises at least an amino acid sequence of an upper part comprising the upper region of beta sheet structure within hemagglutinin HA1 region of influenza A virus and/or a lower part comprising the lower region of beta sheet structure within hemagglutinin HA1 region of influenza A virus.

6. The antigenic peptide according to claim 1, wherein the upper part corresponds to amino acid residues 167 to 187 (SEQ ID NO:4) within the hemagglutinin HA1 region of influenza A virus H3N2 and the lower part corresponds to amino acid residues 225 to 241 (SEQ ID NO: 5) within the hemagglutinin HA1 region of influenza A virus H3N2.

7. The antigenic peptide according to claim 1, wherein the influenza A virus comprises an H1, H2, H3, H4, H5, H6, H7, H8, H9, H10, H11, H12, H13, H14, H15, and/or H16 subtype.

8. The antigenic peptide according to claim 1, wherein the influenza A virus comprises H1N1, H1N1-pdm, H3N2, and/or H5N1 subtype.

9. An isolated nucleic acid molecule comprising a nucleotide sequence of a gene encoding the antigenic peptide according to claim 1.

10. A vaccine against influenza A virus comprising at least one antigenic peptide according to claim 1.

11. A reagent for influenza test comprising at least one antigenic peptide according to claim 1.

12. A reagent kit for influenza test comprising at least one antigenic peptide according to claim 1.

13. A method for selecting an antibody that recognizes an amino acid sequence of an upper region and/or a lower region of beta sheet structure within hemagglutinin HA1 region of an influenza A virus subtype.

14. The method for selecting an antibody according to claim 13, wherein the upper region corresponds to amino acid residues 173 to 181 (SEQ ID NO: 2) within the hemagglutinin HA1 region of influenza A virus H3N2 and the lower region corresponds to amino acid residues 227 to 239 (SEQ ID NO: 3) within the hemagglutinin HA1 region of influenza A virus H3N2.

15. The method for selecting an antibody according to claim 13, wherein the amino acid sequence of the upper region is selected from the group consisting of: SEQ ID NO: 34 (EKEVLVLWG), SEQ ID NO: 2 (KFDKLYIWG), SEQ ID NO: 71(QEDLLVLWG), and the amino acid sequence modified by substitution, insertion, addition and/or deletion of one or two amino acid residue(s) of SEQ IN NO: 34, 2, or 71; and the amino acid sequence of the lower region is selected from the group consisting of: SEQ ID NO: 51 (EGRINYYWTLLEP), SEQ ID NO: 3 (PSRISIYWTIVKP), SEQ ID NO: 82 (SGRMEFFWTILKP), and the amino acid sequence modified by substitution, insertion, addition and/or deletion of one or two amino acid residue(s) of SEQ IN NO: 51, 3, or 82.

16. The method for selecting an antibody according to claim 13, wherein the amino acid sequence of the upper region and/or the lower region is conserved in all influenza A virus subtypes derived from human, swine, or avian hosts.

17. The method for selecting an antibody according to claim 13, wherein the antibody recognizes an amino acid sequence of an upper part comprising the upper region of beta sheet structure within hemagglutinin HA1 region of influenza A virus and/or a lower part comprising the lower region of beta sheet structure within hemagglutinin HA1 region of influenza A virus.

18. The method for selecting an antibody according to claim 17, wherein the upper part corresponds to amino acid residues 167 to 187 (SEQ ID NO: 4) within the hemagglutinin HA1 region of influenza A virus H3N2 and the lower part corresponds to amino acid residues 225 to 241 (SEQ ID NO: 5) within the hemagglutinin HA1 region of influenza A virus H3N2.

19. The method for selecting an antibody or antibodies according to claim 13, wherein the influenza A virus comprises an H1, H2, H3, H4, H5, H6, H7, H8, H9, H10, H11, H12, H13, H14, H15, or H16 subtype.

20. The method for selecting an antibody or antibodies according to claim 13, wherein the influenza A virus comprises H1N1, H1N1 pdm, H3N2, or H5N1 subtype.

21. The method for selecting an antibody or antibodies according to claim 13 comprising incubating an antibody with an antigenic peptide to detect the binding between the antibody and the antigenic peptide, wherein the antigenic peptide comprises the amino acid sequence of the upper region and/or the lower region of beta sheet structure within hemagglutinin HA1 region of an influenza A virus.

22. The method for selecting an antibody according to claim 13 comprising incubating an antibody with an antigenic peptide to detect the binding between the antibody and the antigenic peptide, wherein the antigenic peptide comprises the amino acid sequence of the upper part and/or the lower part of beta sheet structure within hemagglutinin HA1 region of an influenza A virus.

23. The method for selecting an antibody according to claim 13, wherein the selected antibody shows neutralization of the antigenic peptide.

24. The method for selecting an antibody according to claim 13, wherein the selected antibody shows inhibition of hemagglutination (HI).

25. The method for preparing the antibody by using the peptide according to claim 1.


SEQUENCE LISTING (182 sequences)

SEQ ID NO: 1 - protein, 329 aa, Influenza A virus
QKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSACKRRSNNSFFSRLNWLTRLKFKYPALNVTMPNNEKFDKLYIWGVHHPGTDNDQIFLYAQASGRITVSTKRSQQTVIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTR

SEQ ID NO: 2 - protein, 9 aa, Artificial Sequence
  partial peptide of hemagglutinin of Influenza A virus
KFDKLYIWG

SEQ ID NO: 3 - protein, 13 aa, Artificial Sequence
  partial peptide of hemagglutinin of Influenza A virus
PSRISIYWTIVKP

SEQ ID NO: 4 - protein, 21 aa, Artificial Sequence
  partial peptide of hemagglutinin of Influenza A virus
TMPNNEKFDKLYIWGVHHPGT

SEQ ID NO: 5 - protein, 17 aa, Artificial Sequence
  partial peptide of hemagglutinin of Influenza A virus
NIPSRISIYWTIVKPGD

SEQ ID NO: 6 - protein, 20 aa, Artificial Sequence
  partial peptide of hemagglutinin of Influenza A virus
MPNNEKFDKLYIWGVHHPGT

SEQ ID NO: 7 - protein, 21 aa, Artificial Sequence
  partial peptide of hemagglutinin of Influenza A virus
NIPSRISIYWTIVKPGDILLI

SEQ ID NO: 8 - protein, 9 aa, Artificial Sequence
  partial peptide of hemagglutinin of Influenza A virus
EFDKLYIWG

SEQ ID NO: 9 - protein, 9 aa, Artificial Sequence
  partial peptide of hemagglutinin of Influenza A virus
NFDKLYIWG

SEQ ID NO: 10 - protein, 9 aa, Artificial Sequence
  partial peptide of hemagglutinin of Influenza A virus
QFDKLYIWG

SEQ ID NO: 11 - protein, 9 aa, Artificial Sequence
  partial peptide of hemagglutinin of Influenza A virus
NSDKLYIWG

SEQ ID NO: 12 - protein, 9 aa, Artificial Sequence
  partial peptide of hemagglutinin of Influenza A virus
RFDKLYIWG

SEQ ID NO: 13 - protein, 9 aa, Artificial Sequence
  partial peptide of hemagglutinin of Influenza A virus
KFDKLYVWG

SEQ ID NO: 14 - protein, 9 aa, Artificial Sequence
  partial peptide of hemagglutinin of Influenza A virus
KFEKLYIWG

SEQ ID NO: 15 - protein, 9 aa, Artificial Sequence
  partial peptide of hemagglutinin of Influenza A virus
KYDKLYIWG

SEQ ID NO: 16 - protein, 9 aa, Artificial Sequence
  partial peptide of hemagglutinin of Influenza A virus
GFDKLYIWG

SEQ ID NO: 17 - protein, 9 aa, Artificial Sequence
  partial peptide of hemagglutinin of Influenza A virus
DFDKLYIWG

SEQ ID NO: 18 - protein, 9 aa, Artificial Sequence
  partial peptide of hemagglutinin of Influenza A virus
NFDKLYVWG

SEQ ID NO: 19 - protein, 9 aa, Artificial Sequence
  partial peptide of hemagglutinin of Influenza A virus
NFNKLYIWG

SEQ ID NO: 20 - protein, 9 aa, Artificial Sequence
  partial peptide of hemagglutinin of Influenza A virus
DFNKLYIWG

SEQ ID NO: 21 - protein, 13 aa, Artificial Sequence
  partial peptide of hemagglutinin of Influenza A virus
SSRISIYWTIVKP

SEQ ID NO: 22 - protein, 13 aa, Artificial Sequence
  partial peptide of hemagglutinin of Influenza A virus
SSIISIYWTIVKP

SEQ ID NO: 23 - protein, 13 aa, Artificial Sequence
  partial peptide of hemagglutinin of Influenza A virus
SSKISIYWTIVKP

SEQ ID NO: 24 - protein, 13 aa, Artificial Sequence
  partial peptide of hemagglutinin of Influenza A virus
SSRVSIYWTIVKP

SEQ ID NO: 25 - protein, 13 aa, Artificial Sequence
  partial peptide of hemagglutinin of Influenza A virus
SSGISIYWTIVKP

SEQ ID NO: 26 - protein, 13 aa, Artificial Sequence
  partial peptide of hemagglutinin of Influenza A virus
  misc_feature (3)..(3): Xaa can be any naturally occurring amino acid
SSXISIYWTIVKP

SEQ ID NO: 27 - protein, 13 aa, Artificial Sequence
  partial peptide of hemagglutinin of Influenza A virus
SSRISIHWTIVKP

SEQ ID NO: 28 - protein, 13 aa, Artificial Sequence
  partial peptide of hemagglutinin of Influenza A virus
SSRISIYWTIVKS

SEQ ID NO: 29 - protein, 13 aa, Artificial Sequence
  partial peptide of hemagglutinin of Influenza A virus
SSRINIYWTIVKP

SEQ ID NO: 30 - protein, 13 aa, Artificial Sequence
  partial peptide of hemagglutinin of Influenza A virus
PGRISIYWTIVKP

SEQ ID NO: 31 - protein, 13 aa, Artificial Sequence
  partial peptide of hemagglutinin of Influenza A virus
SSRMSIYWTIVKP

SEQ ID NO: 32 - protein, 13 aa, Artificial Sequence
  partial peptide of hemagglutinin of Influenza A virus
SSRISIYWTTVKP

SEQ ID NO: 33 - protein, 13 aa, Artificial Sequence
  partial peptide of hemagglutinin of Influenza A virus
SSRIGIHWTIVKP

SEQ ID NO: 34 - protein, 9 aa, Artificial Sequence
  partial peptide of hemagglutinin of Influenza A virus
EKEVLVLWG

SEQ ID NO: 35 - protein, 9 aa, Artificial Sequence
  partial peptide of hemagglutinin of Influenza A virus
GKEVLVLWG

SEQ ID NO: 36 - protein, 9 aa, Artificial Sequence
  partial peptide of hemagglutinin of Influenza A virus
KKEVLVLWG

SEQ ID NO: 37 - protein, 9 aa, Artificial Sequence
  partial peptide of hemagglutinin of Influenza A virus
EKEVLVMWG

SEQ ID NO: 38 - protein, 9 aa, Artificial Sequence
  partial peptide of hemagglutinin of Influenza A virus
EKEVLILWG

SEQ ID NO: 39 - protein, 9 aa, Artificial Sequence
  partial peptide of hemagglutinin of Influenza A virus
KKEVLVIWG

SEQ ID NO: 40 - protein, 9 aa, Artificial Sequence
  partial peptide of hemagglutinin of Influenza A virus
EKEVLLLWG

SEQ ID NO: 41 - protein, 9 aa, Artificial Sequence
  partial peptide of hemagglutinin of Influenza A virus
DKEVLVLWG

SEQ ID NO: 42 - protein, 9 aa, Artificial Sequence
  partial peptide of hemagglutinin of Influenza A virus
EKKVLVLWG

SEQ ID NO: 43 - protein, 9 aa, Artificial Sequence
  partial peptide of hemagglutinin of Influenza A virus
EKEILVLWG

SEQ ID NO: 44 - protein, 9 aa, Artificial Sequence
  partial peptide of hemagglutinin of Influenza A virus
GKEVLVIWG

SEQ ID NO: 45 - protein, 9 aa, Artificial Sequence
  partial peptide of hemagglutinin of Influenza A virus
EKEVLVLWG

SEQ ID NO: 46 - protein, 9 aa, Artificial Sequence
  partial peptide of hemagglutinin of Influenza A virus
RKEVLVIWG

SEQ ID NO: 47 - protein, 9 aa, Artificial Sequence
  partial peptide of hemagglutinin of Influenza A virus
GKEVLIIWG

SEQ ID NO: 48 - protein, 9 aa, Artificial Sequence
  partial peptide of hemagglutinin of Influenza A virus
GREVLVLWG

SEQ ID NO: 49 - protein, 9 aa, Artificial Sequence
  partial peptide of hemagglutinin of Influenza A virus
GKKVLVLWG

SEQ ID NO: 50 - protein, 9 aa, Artificial Sequence
  partial peptide of hemagglutinin of Influenza A virus
EKEVLVLWA

SEQ ID NO: 51 - protein, 13 aa, Artificial Sequence
  partial peptide of hemagglutinin of Influenza A virus
EGRINYYWTLLEP

SEQ ID NO: 52 - protein, 13 aa, Artificial Sequence
  partial peptide of hemagglutinin of Influenza A virus
AGRINYYWTLLEP

SEQ ID NO: 53 - protein, 13 aa, Artificial Sequence
  partial peptide of hemagglutinin of Influenza A virus
AGRMNYYWTLLEP

SEQ ID NO: 54 - protein, 13 aa, Artificial Sequence
  partial peptide of hemagglutinin of Influenza A virus
AGRMNYYWTLLKP

SEQ ID NO: 55 - protein, 13 aa, Artificial Sequence
  partial peptide of hemagglutinin of Influenza A virus
HGRMNYYWTLLEP

SEQ ID NO: 56 - protein, 13 aa, Artificial Sequence
  partial peptide of hemagglutinin of Influenza A virus
AGRMNYYWTLVEP

SEQ ID NO: 57 - protein, 13 aa, Artificial Sequence
  partial peptide of hemagglutinin of Influenza A virus
PGRMNYYWTLLEP

SEQ ID NO: 58 - protein, 13 aa, Artificial Sequence
  partial peptide of hemagglutinin of Influenza A virus
EGRISYYWTLLEP

SEQ ID NO: 59 - protein, 13 aa, Artificial Sequence
  partial peptide of hemagglutinin of Influenza A virus
EGRINYHWTLLEP

SEQ ID NO: 60 - protein, 13 aa, Artificial Sequence
  partial peptide of hemagglutinin of Influenza A virus
AGRMNYYWTLIEP

SEQ ID NO: 61 - protein, 13 aa, Artificial Sequence
  partial peptide of hemagglutinin of Influenza A virus
AGRMNYYWTLLDQ

SEQ ID NO: 62 - protein, 13 aa, Artificial Sequence
  partial peptide of hemagglutinin of Influenza A virus
AGRMNYYWTLVEP

SEQ ID NO: 63 - protein, 13 aa, Artificial Sequence
  partial peptide of hemagglutinin of Influenza A virus
AGRMNYYWTLLEP

SEQ ID NO: 64 - protein, 13 aa, Artificial Sequence
  partial peptide of hemagglutinin of Influenza A virus
AGRMNYHWTLIEP

SEQ ID NO: 65 - protein, 13 aa, Artificial Sequence
  partial peptide of hemagglutinin of Influenza A virus
TGRMNYYWTLVEP

SEQ ID NO: 66 - protein, 13 aa, Artificial Sequence
  partial peptide of hemagglutinin of Influenza A virus
AGRMDYYWTLVEP

SEQ ID NO: 67 - protein, 13 aa, Artificial Sequence
  partial peptide of hemagglutinin of Influenza A virus
AGRINYYWTLLDQ

SEQ ID NO: 68 - protein, 13 aa, Artificial Sequence
  partial peptide of hemagglutinin of Influenza A virus
TGRMNYYWTLLEP

SEQ ID NO: 69 - protein, 13 aa, Artificial Sequence
  partial peptide of hemagglutinin of Influenza A virus
EGRMNYYWTLVEP

SEQ ID NO: 70 - protein, 13 aa, Artificial Sequence
  partial peptide of hemagglutinin of Influenza A virus
EGRMNYYWTLIEP

SEQ ID NO: 71 - protein, 9 aa, Artificial Sequence
  partial peptide of hemagglutinin of Influenza A virus
QEDLLVLWG

SEQ ID NO: 72 - protein, 9 aa, Artificial Sequence
  partial peptide of hemagglutinin of Influenza A virus
QEDLLVMWG

SEQ ID NO: 73 - protein, 9 aa, Artificial Sequence
  partial peptide of hemagglutinin of Influenza A virus
QEDLLILWG

SEQ ID NO: 74 - protein, 9 aa, Artificial Sequence
  partial peptide of hemagglutinin of Influenza A virus
QENLLILWG

SEQ ID NO: 75 - protein, 9 aa, Artificial Sequence
  partial peptide of hemagglutinin of Influenza A virus
QEDLLVIWG

SEQ ID NO: 76 - protein, 9 aa, Artificial Sequence
  partial peptide of hemagglutinin of Influenza A virus
REDLLVLWG

SEQ ID NO: 77 - protein, 9 aa, Artificial Sequence
  partial peptide of hemagglutinin of Influenza A virus
VEDLLIIWG

SEQ ID NO: 78 - protein, 9 aa, Artificial Sequence
  partial peptide of hemagglutinin of Influenza A virus
KEDLLILWG

SEQ ID NO: 79 - protein, 9 aa, Artificial Sequence
  partial peptide of hemagglutinin of Influenza A virus
PEDLLVLWG

SEQ ID NO: 80 - protein, 9 aa, Artificial Sequence
  partial peptide of hemagglutinin of Influenza A virus
QEDLLIMWG

SEQ ID NO: 81 - protein, 9 aa, Artificial Sequence
  partial peptide of hemagglutinin of Influenza A virus
MEDLLILWG

SEQ ID NO: 82 - protein, 13 aa, Artificial Sequence
  partial peptide of hemagglutinin of Influenza A virus
SGRMEFFWTILKP

SEQ ID NO: 83 - protein, 13 aa, Artificial Sequence
  partial peptide of hemagglutinin of Influenza A virus
SGRMEFFWTILNP

SEQ ID NO: 84 - protein, 13 aa, Artificial Sequence
  partial peptide of hemagglutinin of Influenza A virus
SGRMDFFWTILKP

SEQ ID NO: 85 - protein, 13 aa, Artificial Sequence
  partial peptide of hemagglutinin of Influenza A virus
SGRMEFFWTILKS

SEQ ID NO: 86 - protein, 13 aa, Artificial Sequence
  partial peptide of hemagglutinin of Influenza A virus
SGRMEFFWAILKP

SEQ ID NO: 87 - protein, 13 aa, Artificial Sequence
  partial peptide of hemagglutinin of Influenza A virus
NGRMEFFWTILKP

SEQ ID NO: 88 - protein, 13 aa, Artificial Sequence
  partial peptide of hemagglutinin of Influenza A virus
SGRIEFFWTILKS

SEQ ID NO: 89 - protein, 13 aa, Artificial Sequence
  partial peptide of hemagglutinin of Influenza A virus
SGRIEFFWTILKP

SEQ ID NO: 90 - protein, 13 aa, Artificial Sequence
  partial peptide of hemagglutinin of Influenza A virus
SGRMDFFWTMLRP

SEQ ID NO: 91 - protein, 13 aa, Artificial Sequence
  partial peptide of hemagglutinin of Influenza A virus
SGRMEFFRTILKP

SEQ ID NO: 92 - protein, 13 aa, Artificial Sequence
  partial peptide of hemagglutinin of Influenza A virus
SGRIDFFWTILKP

SEQ ID NO: 93 - protein, 13 aa, Artificial Sequence
  partial peptide of hemagglutinin of Influenza A virus
SGRVEFFWTILKS

SEQ ID NO: 94 - protein, 13 aa, Artificial Sequence
  partial peptide of hemagglutinin of Influenza A virus
SGRMDFFWTMLKP

SEQ ID NO: 95 - protein, 13 aa, Artificial Sequence
  partial peptide of hemagglutinin of Influenza A virus
SGRMEFFWTILRP

SEQ ID NO: 96 - protein, 13 aa, Artificial Sequence
  partial peptide of hemagglutinin of Influenza A virus
SGRMEFFWTILEP

SEQ ID NO: 97 - protein, 26 aa, Artificial Sequence
  partial peptide of hemagglutinin of Influenza A virus
NNESFNWTGVTQNGTSSACKRRSNNS

SEQ ID NO: 98 - protein, 26 aa, Artificial Sequence
  partial peptide of hemagglutinin of Influenza A virus
NNESFNWTGVTQNGTSSACIRRSNNS

SEQ ID NO: 99 - protein, 26 aa, Artificial Sequence
  partial peptide of hemagglutinin of Influenza A virus
NNESFNWTGVAQNGTSSACKRRSDKS

SEQ ID NO: 100 - protein, 26 aa, Artificial Sequence
  partial peptide of hemagglutinin of Influenza A virus
NNESFNWTGVAQNGTSSACKRRSNKS

SEQ ID NO: 101 - protein, 26 aa, Artificial Sequence
  partial peptide of hemagglutinin of Influenza A virus
NNESFDWTGVTQNGTSSACKRRSNKS

SEQ ID NO: 102 - protein, 26 aa, Artificial Sequence
  partial peptide of hemagglutinin of Influenza A virus
NNESFNWTGVAQNGTSSACKRRSIKS

SEQ ID NO: 103 - protein, 26 aa, Artificial Sequence
  partial peptide of hemagglutinin of Influenza A virus
NNESFNWTGVTQNGTSSACKRRSNKS

SEQ ID NO: 104 - protein, 26 aa, Artificial Sequence
  partial peptide of hemagglutinin of Influenza A virus
INEDFNWTGVAQDGKSYACKRGSVNS

SEQ ID NO: 105 - protein, 26 aa, Artificial Sequence
  partial peptide of hemagglutinin of Influenza A virus
NNESFNWTGVTQNGTSSACKRRSNSS

SEQ ID NO: 106 - protein, 26 aa, Artificial Sequence
  partial peptide of hemagglutinin of Influenza A virus
NNESFNWTGVAQNGTSYACKRRSIKS

SEQ ID NO: 107 - protein, 9 aa, Artificial Sequence
  partial peptide of hemagglutinin of Influenza A virus
THLKFKYPA

SEQ ID NO: 108 - protein, 9 aa, Artificial Sequence
  partial peptide of hemagglutinin of Influenza A virus
HQLKYKYPA

SEQ ID NO: 109 - protein, 9 aa, Artificial Sequence
  partial peptide of hemagglutinin of Influenza A virus
THLKYKYPA

SEQ ID NO: 110 - protein, 9 aa, Artificial Sequence
  partial peptide of hemagglutinin of Influenza A virus
HKLEYKYPA

SEQ ID NO: 111 - protein, 9 aa, Artificial Sequence
  partial peptide of hemagglutinin of Influenza A virus
THSKFKYPA

SEQ ID NO: 112 - protein, 9 aa, Artificial Sequence
  partial peptide of hemagglutinin of Influenza A virus
TKSGSTYPV

SEQ ID NO: 113 - protein, 9 aa, Artificial Sequence
  partial peptide of hemagglutinin of Influenza A virus
HQLKYRYPA

SEQ ID NO: 114 - protein, 9 aa, Artificial Sequence
  partial peptide of hemagglutinin of Influenza A virus
YKSGSTYPV

SEQ ID NO: 115 - protein, 9 aa, Artificial Sequence
  partial peptide of hemagglutinin of Influenza A virus
HESEYKYPA

SEQ ID NO: 116 - protein, 9 aa, Artificial Sequence
  partial peptide of hemagglutinin of Influenza A virus
YESESKYPV

SEQ ID NO: 117 - protein, 12 aa, Artificial Sequence
  partial peptide of hemagglutinin of Influenza A virus
GTDSDQISLYAQ

SEQ ID NO: 118 - protein, 12 aa, Artificial Sequence
  partial peptide of hemagglutinin of Influenza] A virus
GTDNDQIFLYAQ

SEQ ID NO: 119 - protein, 12 aa, Artificial Sequence
  partial peptide of hemagglutinin of Influenza A virus
STDSDQISLYAQ

SEQ ID NO: 120 - protein, 12 aa, Artificial Sequence
  partial peptide of hemagglutinin of Influenza A virus
GTDNDQISLYAQ

SEQ ID NO: 121 - protein, 12 aa, Artificial Sequence
  partial peptide of hemagglutinin of Influenza A virus
STDSDQTSLYAQ

SEQ ID NO: 122 - protein, 12 aa, Artificial Sequence
  partial peptide of hemagglutinin of Influenza A virus
STDSDQTSLYVR

SEQ ID NO: 123 - protein, 12 aa, Artificial Sequence
  partial peptide of hemagglutinin of Influenza A virus
STDSDQTSLYVQ

SEQ ID NO: 124 - protein, 12 aa, Artificial Sequence
  partial peptide of hemagglutinin of Influenza A virus
GTYNDQISLYAQ

SEQ ID NO: 125 - protein, 12 aa, Artificial Sequence
  partial peptide of hemagglutinin of Influenza A virus
STNQEQTSLYVQ

SEQ ID NO: 126 - protein, 12 aa, Artificial Sequence
  partial peptide of hemagglutinin of Influenza A virus
GTNNDQISLYAQ

SEQ ID NO: 127 - protein, 8 aa, Artificial Sequence
  partial peptide of hemagglutinin of Influenza A virus
GICDSPHQ

SEQ ID NO: 128 - protein, 8 aa, Artificial Sequence
  partial peptide of hemagglutinin of Influenza A virus
RICDSPHQ

SEQ ID NO: 129 - protein, 8 aa, Artificial Sequence
  partial peptide of hemagglutinin of Influenza A virus
EICDSPHQ

SEQ ID NO: 130 - protein, 8 aa, Artificial Sequence
  partial peptide of hemagglutinin of Influenza A virus
RICDSPHR

SEQ ID NO: 131 - protein, 8 aa, Artificial Sequence
  partial peptide of hemagglutinin of Influenza A virus
KICNNPHR

SEQ ID NO: 132 - protein, 8 aa, Artificial Sequence
  partial peptide of hemagglutinin of Influenza A virus
RICNSPHQ

SEQ ID NO: 133 - protein, 8 aa, Artificial Sequence
  partial peptide of hemagglutinin of Influenza A virus
KICDNPHR

SEQ ID NO: 134 - protein, 8 aa, Artificial Sequence
  partial peptide of hemagglutinin of Influenza A virus
GICDSPYQ

SEQ ID NO: 135 - protein, 8 aa, Artificial Sequence
  partial peptide of hemagglutinin of Influenza A virus
GICNSPHQ

SEQ ID NO: 136 - protein, 8 aa, Artificial Sequence
  partial peptide of hemagglutinin of Influenza A virus
EICNSPHQ

SEQ ID NO: 137 - protein, 5 aa, Artificial Sequence
  partial peptide of hemagglutinin of Influenza A virus
GKCNS

SEQ ID NO: 138 - protein, 5 aa, Artificial Sequence
  partial peptide of hemagglutinin of Influenza A virus
GNCSS

SEQ ID NO: 139 - protein, 5 aa, Artificial Sequence
  partial peptide of hemagglutinin of Influenza A virus
GTCSS

SEQ ID NO: 140 - protein, 5 aa, Artificial Sequence
  partial peptide of hemagglutinin of Influenza A virus
DNCNS

SEQ ID NO: 141 - protein, 5 aa, Artificial Sequence
  partial peptide of hemagglutinin of Influenza A virus
DTCIS

SEQ ID NO: 142 - protein, 5 aa, Artificial Sequence
  partial peptide of hemagglutinin of Influenza A virus
GNCNS

SEQ ID NO: 143 - protein, 5 aa, Artificial Sequence
  partial peptide of hemagglutinin of Influenza A virus
DKCNS

SEQ ID NO: 144 - protein, 5 aa, Artificial Sequence
  partial peptide of hemagglutinin of Influenza A virus
GTCIS

SEQ ID NO: 145 - protein, 5 aa, Artificial Sequence
  partial peptide of hemagglutinin of Influenza A virus
GKCKS

SEQ ID NO: 146 - protein, 5 aa, Artificial Sequence
  partial peptide of hemagglutinin of Influenza A virus
VKCNS

SEQ ID NO: 147 - protein, 13 aa, Artificial Sequence
  partial peptide of hemagglutinin of Influenza A virus
KRSQQTVIPNIGS

SEQ ID NO: 148 - protein, 13 aa, Artificial Sequence
  partial peptide of hemagglutinin of Influenza A virus
KRSQQTVIPNIGF

SEQ ID NO: 149 - protein, 13 aa, Artificial Sequence
  partial peptide of hemagglutinin of Influenza A virus
RRSQQTIIPNIGS

SEQ ID NO: 150 - protein, 13 aa, Artificial Sequence
  partial peptide of hemagglutinin of Influenza A virus
KRSQQTVIPDIGY

SEQ ID NO: 151 - protein, 13 aa, Artificial Sequence
  partial peptide of hemagglutinin of Influenza A virus
KRSQQTIIPNIGS

SEQ ID NO: 152 - protein, 13 aa, Artificial Sequence
  partial peptide of hemagglutinin of Influenza A virus
KRSQQTVTPNIGS

SEQ ID NO: 153 - protein, 13 aa, Artificial Sequence
  partial peptide of hemagglutinin of Influenza A virus
KRSQQTIIPNVGS

SEQ ID NO: 154 - protein, 13 aa, Artificial Sequence
  partial peptide of hemagglutinin of Influenza A virus
KRSQQTVIPNIGP

SEQ ID NO: 155 - protein, 13 aa, Artificial Sequence
  partial peptide of hemagglutinin of Influenza A virus
KRSQQTVIPNVGS

SEQ ID NO: 156 - protein, 13 aa, Artificial Sequence
  partial peptide of hemagglutinin of Influenza A virus
RRSQQTVIPNIGS

SEQ ID NO: 157 - protein, 22 aa, Artificial Sequence
  partial peptide of hemagglutinin of Influenza A virus
ENCTLIDALLGDPQCDGFQNKK

SEQ ID NO: 158 - protein, 22 aa, Artificial Sequence
  partial peptide of hemagglutinin of Influenza A virus
ENCTLIDALLGDPHCDGFQNKE

SEQ ID NO: 159 - protein, 22 aa, Artificial Sequence
  partial peptide of hemagglutinin of Influenza A virus
KNCTLIDALLGDPHCDGFQNKE

SEQ ID NO: 160 - protein, 22 aa, Artificial Sequence
  partial peptide of hemagglutinin of Influenza A virus
KNCTLIDALLGDPHCDGFQNEK

SEQ ID NO: 161 - protein, 22 aa, Artificial Sequence
  partial peptide of hemagglutinin of Influenza A virus
IDCTLIDALLGDPHCDVFQNET

SEQ ID NO: 162 - protein, 22 aa, Artificial Sequence
  partial peptide of hemagglutinin of Influenza A virus
ENCTLIDALLGDPQCDGFQNKN

SEQ ID NO: 163 - protein, 22 aa, Artificial Sequence
  partial peptide of hemagglutinin of Influenza A virus
ENCTLIDALLGDPHCDGFQNKK

SEQ ID NO: 164 - protein, 22 aa, Artificial Sequence
  partial peptide of hemagglutinin of Influenza A virus
IDCTLIDALLGDPHCDGFQNET

SEQ ID NO: 165 - protein, 22 aa, Artificial Sequence
  partial peptide of hemagglutinin of Influenza A virus
INCTLIDALLGDPHCDGFQNEK

SEQ ID NO: 166 - protein, 22 aa, Artificial Sequence
  partial peptide of hemagglutinin of Influenza A virus
ENCTLIDALLGDPHCDSFQNKE

SEQ ID NO: 167 - protein, 15 aa, Artificial Sequence
  partial peptide of hemagglutinin of Influenza A virus
TMPNNEKFDKLYIWG

SEQ ID NO: 168 - protein, 15 aa, Artificial Sequence
  partial peptide of hemagglutinin of Influenza A virus
PNNEKFDKLYIWGVH

SEQ ID NO: 169 - protein, 15 aa, Artificial Sequence
  partial peptide of hemagglutinin of Influenza A virus
NEKFDKLYIWGVHHP

SEQ ID NO: 170 - protein, 15 aa, Artificial Sequence
  partial peptide of hemagglutinin of Influenza A virus
KFDKLYIWGVHHPGT

SEQ ID NO: 171 - protein, 15 aa, Artificial Sequence
  partial peptide of hemagglutinin of Influenza A virus
NIPSRISIYWTIVKP

SEQ ID NO: 172 - protein, 15 aa, Artificial Sequence
  partial peptide of hemagglutinin of Influenza A virus
PSRISIYWTIVKPGD

SEQ ID NO: 173 - DNA, 27 bp, Artificial Sequence
  nucleotide sequence coding for partial peptide of hemagglutinin of Influenza A virus
aaatttgacaaattgtacatttggggg

SEQ ID NO: 174 - DNA, 39 bp, Artificial Sequence
  nucleotide sequence coding for partial peptide of hemagglutinin of Influenza A virus
tccagcagaataagcatctattggacaatagtaaaaccg

SEQ ID NO: 175 - DNA, 39 bp, Artificial Sequence
  nucleotide sequence coding for partial peptide of hemagglutinin of Influenza A virus
tctagtagaataagcatctattggacaatagtaaaaccg

SEQ ID NO: 176 - DNA, 39 bp, Artificial Sequence
  nucleotide sequence coding for partial peptide of hemagglutinin of Influenza A virus
cccagcagaataagcatctattggacaatagtaaaaccg

SEQ ID NO: 177 - protein, 9 aa, Artificial Sequence
  synthesized peptide based on hemagglutinin of Influenza A virus
CKEVLVLWG

SEQ ID NO: 178 - protein, 13 aa, Artificial Sequence
  synthesized peptide based on hemagglutinin of Influenza A virus
CGRINYYWTLLEP

SEQ ID NO: 179 - protein, 9 aa, Artificial Sequence
  synthesized peptide based on hemagglutinin of Influenza A virus
CFDKLYIWG

SEQ ID NO: 180 - protein, 13 aa, Artificial Sequence
  synthesized peptide based on hemagglutinin of Influenza A virus
CSRISIYWTIVKP

SEQ ID NO: 181 - protein, 10 aa, Artificial Sequence
  synthesized peptide based on hemagglutinin of Influenza A virus
QEDLLVLWGC

SEQ ID NO: 182 - protein, 14 aa, Artificial Sequence
  synthesized peptide based on hemagglutinin of Influenza A virus
SGRMEFFWTILKPC